SEQUENCE LISTING (9 sequences)

SEQ ID NO: 1 - protein, 323 aa, Homo sapiens
  human leukocyte surface antigen CD47 isoform 1 precursor
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 305 aa, Homo sapiens
  human CD47 protein
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 3 - DNA, 5292 bp, Homo sapiens
  human CD47 transcript variant 1 (mRNA)
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 4 - DNA, 5288 bp, Homo sapiens
  human CD47 transcript variant 2 (mRNA)
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 5 - protein, 324 aa, Mus musculus
  leukocyte surface antigen CD47 isoform 4 precursor
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTAFNTDQGSACSYEEEKGGCKLVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 6 - protein, 312 aa, Homo sapiens
  leukocyte surface antigen CD47 isoform 3
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 7 - protein, 293 aa, Homo sapiens
  leukocyte surface antigen CD47 isoform X2
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 8 - DNA, 5008 bp, Homo sapiens
  PREDICTED Homo sapiens CD47 molecule (CD47), transcript variant X11, mRNA
gtgcgcgcggccgtgcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagcta

SEQ ID NO: 9 - DNA, 5259 bp, Artificial Sequence
  Homo sapiens CD47 molecule (CD47), transcript variant 3, mRNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

(19) United States
(12) Patent Application Publication
(10) Pub. No.: US 2023/0414664 A1
(43) Pub. Date: Dec. 28, 2023

(54) GENETIC MODIFICATIONS FOR XENOTRANSPLANTATION

(71) Applicant: THE TRUSTEES OF COLUMBIA UNIVERSIT IN THE CITY OF NEW YORK, New Yok, NY (US)

(72) Inventors: Megan Sykes, New York, NY (US); Robert J. Hawley, River Edge, NJ (US); Kazuhiko Yamada, Fort Lee, NJ (US); David H. Sachs, Auburndale, MA (US)

(21) Appl. No.: 18/024,701

(22) PCT Filed: Sep. 3, 2021
(86) PCT No.: PCT/US2021/049019

§ 371 Date: Mar. 3, 2023

Related U.S. Application Data
(60) Provisional application No. 63/108,986, filed on Nov. 3, 2020.
(60) Provisional application No. 63/075,285, filed on Sep. 7, 2020.

(51) Int. Cl. A61K 35/22; A61K 35/28; A61K 38/17; A61K 35/39; A61K 45/06; A61P 13/02; A61L 27/36; A61L 27/38; A01K 67/027

(52) U.S. Cl. CPC A61K 35/22; A01K 2267/025; A61K 38/1774; A61K 35/39; A61K 45/06; A61P 13/02; A61L 27/3604; A61L 27/3679; A61L 27/3804; A61L 27/3882; A01K 67/0278; A61L 2430/26; A01K 2227/108; A01K 2207/15; A01K 2217/052; A61K 35/28

(57) ABSTRACT

Provided herein are recombinant miniature swine expressing human CD47 in a tissue specific fashion. Also provided are kidneys isolated from a miniature swine, wherein the glomeruli of the kidney express human CD47 at a level higher than the level of human CD47 expression in the tubules of the kidney. Furthermore, provided herein are methods of transplanting such swine kidneys with glomeruli-specific expression of human CD47 from recombinant miniature swine into human recipients. In certain aspects, provided herein are methods of transplantation comprising transplanting hematopoietic stem cells expressing human CD47 from a first donor animal (e.g., a miniature swine) and a kidney expressing human CD47 in the glomeruli from a second donor animal (e g., a miniature swine) to a recipient (e.g., a human recipient).

### 2. CROSS-REFERENCE TO RELATED APPLICATIONS

This application claims the benefit of U.S. Ser. No. 63/075,285 filed Sep. 7, 2020, and U.S. Ser. No. 63/108,986 filed Nov. 3, 2020, the disclosure of each of which is incorporated by reference herein in its entirety.

### 1. GOVERNMENT LICENSE RIGHTS

This invention was made with government support under grant number P01 AI045897 awarded by National Institute of Allergy and Infectious Diseases (NIAID), National Institutes of Health (NIH). The government has certain rights in the invention.

### 3. REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Aug. 26, 2021, is named 14648-015-228_Sequence_Listing.txt and is 42,743 bytes in size.

### 4. FIELD

Provided herein are recombinant miniature swine (e.g., GalT knockout miniature swine) expressing human CD47 in a tissue specific fashion. Also provided are kidneys isolated from a miniature swine, wherein the glomeruli of the kidney express human CD47 at a level higher than the level of human CD47 expression in the tubules of the kidney. Furthermore, provided herein are methods of transplanting such swine kidneys with glomeruli-specific expression of human CD47 from recombinant miniature swine into human recipients. In certain aspects, provided herein are methods of transplantation comprising transplanting hematopoietic stem cells expressing human CD47 from a first donor animal (e.g., a miniature swine) and a kidney expressing human CD47 in the glomeruli from a second donor animal (e.g., a miniature swine) to a recipient (e.g., a human recipient).

### 5. BACKGROUND

The severe shortage of allogeneic donors currently limits the number of organ transplants performed. This supply-demand disparity may be corrected by the use of organs from other species (xenografts). In view of the ethical issues and impracticalities associated with the use of non-human primates, pigs are considered the most suitable donor species for humans. In addition to organ size and physiologic similarities to humans, the ability to rapidly breed and inbreed pigs makes them particularly amenable to genetic modifications that could improve their ability to function as graft donors to humans. See, e.g, Sachs (1994), Path. Biol. 42:217-219 and Piedrahita et al. (2004), Am. J. Transplant, 4 Suppl. 6:43-50.

Although transplantation coupled with non-specific immunosuppressive therapy is associated with high early graft acceptance rates, a major limitation to the success of clinical organ transplantation has been late graft loss, due largely to chronic rejection of the transplant. Immune tolerance is therefore a major goal in transplantation and will be even more important for successful clinical xenotransplantation, as the level of life-long immunosuppression required to prevent xenograft rejection can be too toxic to be acceptable. In addition, no markers have been identified to reliably indicate whether or not immunological tolerance has been achieved in patients, resulting in an absence of laboratory parameters upon which to base immunosuppression withdrawal.

Therefore, goals in xenotransplantation include achievement of tolerance. This could be achieved by xenogeneic thymic transplantation or by optimizing the durability of mixed chimeric cells originated from the donor animal after they are transplanted into a xenogeneic recipient, as well as maintaining the health and viability of the donor animal.

Mixed chimerism can induce tolerance to the donor at the level of T cells, B cells and natural killer (NK) cells in the recipient. See, e.g., Griesemer et al. (2014), Immunol. Rev. 258: 241-258; Sachs et al. (2014), Cold Spring Harb. Perspect. Med. 4:a015529.

CD47, also known as integrin-associated protein (IAP), is a ubiquitously expressed 50-kDa cell surface glycoprotein and serves as a ligand for signal regulatory protein (SIRP)α, (also known as CD172a, and SHPS-1). See, e.g., Brown (2002), Curr. Opin. Cell. Biol., 14:603-7; and Brown and Frazier (2001), Trends Cell Biol., 111130-5. CD47 and SIRPα, constitute a cell-cell communication system that plays important roles in a variety of cellular processes including cell migration, adhesion of B cells, and T cell activation. See, e.g., Liu et al. (2002), J. Biol. Chem. 277: 10028; Motegi et al. (2003), EMBO 122:2634; Yoshida et al. (2002), J. lmmunol. 168:3213; and Latour et al. (2001), J. lmmunol. 167:2547. In addition, the CD47-SIRPα system is implicated in negative regulation of phagocytosis by macrophages. CD47 on the surface of some cell types (i.e., erythrocytes, platelets or leukocytes) inhibited phagocytosis by macrophages. The role of CD47-SIRPα interaction in the inhibition of phagocytosis has been illustrated by the observation that primary, wild-type mouse macrophages rapidly phagocytose unopsonized red blood cells (RBCs) obtained from CD47-deficient mice but not those from wild-type mice. See, e.g., Oldenborg et al. (2000), Science 288:2051. It has also been reported that through its receptors, SIRPα, CD47 inhibits both Fcγ and complement receptor mediated phagocytosis. See, e.g., Oldenborg et al. (2001), J. Exp. Med. 193:855.

CD47KO cells are vigorously rejected by macrophages after infusion into syngeneic wild-type (WT) mice, demonstrating that CD47 provides a “don't eat me” signal to macrophages. See, e.g., Oldenborg P A, et al. (2000), Science, 288:2051-4; and Wang et al. (2007), Proc Natl Acad Sci U S A. 104:13744. Xenotransplantation using pigs as the transplant source has the potential to resolve the severe shortage of human organ donors, a major limiting factor in clinical transplantation. See, e.g., Yang et al. (2007), Nature Reviews Immunology. 7:519-31. The strong rejection of xenogeneic cells by macrophages (see, e.g., Abe (2002), The Journal of Immunology 168:621) is largely caused by the lack of functional interaction between donor CD47 and recipient SIRPα (see, e.g., Wang et al. (2007), Blood; 109:836-42; Ide et al. (2007), Proc Natl Acad Sci USA 104:5062-6; and Navarro-Alvarez (2014), Cell transplantation, 23:345-54), leading to the development of human CD47 transgenic pigs (see, e.g., Tena et al. (2017), Transplantation 101:316-21; and Nomura et al. (2020), Xenotransplantation 2020; 27:e12549). In addition to macrophages, a sub-population of DCs also express SIRPα (see, e.g., Wang et al. (2007), Proc Natl Acad Sci U S A. 104:13744-9; and Guilliams et al. (2016), Immunity. 45:669-84). CD47-SIRPα signaling also inhibits DC activation and their ability to prime T cells, and plays an important role in induction of T cell tolerance by donor-specific transfusion (DST) or hepatocyte transplantation. See, e.g., Wang et al. (2007), Proc Natl Acad Sci U S A. 104:13744-9; Wang et al. (2014), Cell transplantation 23:355-63; and Zhang et al. (2016), Sci Rep. 6:26839.

### 6. SUMMARY

In one aspect, provided herein is a method for preventing or reducing the severity of proteinuria in a kidney transplant recipient, wherein the method comprises: (i) transplanting into the recipient a kidney, wherein the kidney is obtained from an alpha-1,3 galactosyltransferase-deficient miniature swine and glomeruli of the kidney express human CD47 at levels sufficient to prevent or reduce the severity of proteinuria in the recipient; and (ii) transplanting into the recipient porcine hematopoietic stem cells, wherein the porcine hematopoietic stem cells express human CD47 and are obtained from an alpha-1,3 galactosyltransferase-deficient miniature swine.

In some embodiments, the glomeruli of the kidney express human CD47 at a level higher than the level of human CD47 expression in the tubules of the kidney. In some embodiments, the glomeruli of the kidney express human CD47 at a level 2 times to 10 times higher than the level of human CD47 expression in the tubules of the kidney. In some embodiments, the alpha-1,3 galactosyltransferase-deficient miniature swine is a MHC-inbred Columbia/Sachs miniature swine. In some embodiments, the level of human CD47 expression is measured by real-time polymerase chain reaction.

In some embodiments, the recipient is a mammal. In some embodiments, the recipient is a human.

In some embodiments, the porcine hematopoietic stem cells are obtained from bone marrow, peripheral blood, umbilical cord blood, or fetal liver cells.

In some embodiments, the human CD47 is expressed under the same regulatory elements as the endogenous porcine CD47. In some embodiments, the human CD47 replaces an endogenous porcine CD47 in the alpha-1,3 galactosyltransferase-deficient miniature swine. In some embodiments, the human CD47 is expressed under a glomerulus-specific promoter. In some embodiments, the glomerulus-specific promoter is nephrin.

In some embodiments, the proteinuria is renal proteinuria. In some embodiments, the proteinuria is reduced to less than 3 g per 24 hours. In some embodiments, the proteinuria is reduced to 500 mg per 24 hours. In some embodiments, the proteinuria is reduced to 300 mg per 24 hours. In some embodiments, the proteinuria is reduced to 150 mg per 24 hours. In some embodiments, the proteinuria resolves within two weeks of the transplant. In some embodiments, the proteinuria resolves within one month of the transplant. In some embodiments, the proteinuria resolves within two months of the transplant. In some embodiments, the proteinuria resolves within four months of the transplant.

In some embodiments, the kidney is a thymokidney.

In another aspect, provided herein is a kidney isolated from a miniature swine, wherein the glomeruli of the kidney express human CD47 at a level higher than the level of human CD47 expression in the tubules of the kidney. In some embodiments, the glomeruli of the kidney express human CD47 at a level 2 times to 10 times higher than the level of human CD47 expression in the tubules of the kidney. In some embodiments, the level of human CD47 expression is measured by real-time polymerase chain reaction. In some embodiments, the human CD47 is expressed under the same regulatory elements as the endogenous porcine CD47. In some embodiments, the human CD47 is expressed under a glomerulus-specific promoter. In some embodiments, the glomerulus-specific promoter is nephrin. In some embodiments, the kidney is a thymokidney. In some embodiments, the miniature swine is an alpha-1,3 galactosyltransferase-deficient miniature swine. In some embodiments, the alpha-1,3 galactosyltransferase-deficient miniature swine is a MHC-inbred Columbia/Sachs miniature swine.

In another aspect, provided herein is a method of transplanting a kidney from a miniature swine into a human recipient, wherein the method comprises (i) transplanting bone marrow from a first miniature swine to the recipient via intra-bone transplantation; and (ii) transplanting a kidney from a second miniature swine to the recipient. In some embodiments, said second step of transplanting a kidney from a second miniature swine is carried out at least 28 days after first step of transplanting bone marrow from a first miniature swine.

In some embodiments, the bone marrow from the first miniature swine expresses human CD47. In some embodiments, the kidney from the second miniature swine expresses human CD47. In some embodiments, the bone marrow from the first miniature swine and the kidney from the second miniature swine express human CD47.

In some embodiments, the human CD47 is expressed under the same regulatory elements as the endogenous porcine CD47. In some embodiments, the human CD47 is expressed under a glomerulus-specific promoter. In some embodiments, the glomerulus-specific promoter is nephrin.

In some embodiments, the bone marrow and the kidney are from the same miniature swine. In some embodiments, the first miniature swine and the second miniature swine are from the same, highly inbred herd of miniature swine. In some embodiments, the first miniature swine and the second miniature swine are alpha-1,3 galactosyltransferase-deficient miniature swine. In some embodiments, the alpha-1,3 galactosyltransferase-deficient miniature swine are MHC-inbred Columbia/Sachs miniature swine. In some embodiments, the first miniature swine and the second miniature swine are genetically matched miniature swine. In some embodiments, the first and the second miniature swine are MHC matched.

In some embodiments, the method further comprises administration of one or more additional treatments to the recipient. In some embodiments, the one or more additional treatment is selected from the group comprising total body irradiation, thymic irradiation, rituximab, anti-thymocyte globulin (ATG), tacrolimus, mycophenolate mofetil (MMF), anti-CD154 antibodies, cobra venom factor (CVF), heparin, prostacyclin, recombinant porcine cytokines, porcine stem cell factor (pCSF), porcine interleukin-3 (pIL-3), ganciclovir, methylprednisolone, anti-IL6 receptor antibodies and anti-CD40 antibodies. In some embodiments, the method further comprises transplanting islet of Langerhans cells from a miniature swine to the recipient.

In another aspect, provided herein is a xenograft from a non-human species, wherein the xenograft comprises: (a) a kidney; and (b) islet of Langerhans cells, wherein the kidney comprises glomeruli that express human CD47 at a level higher than the level of human CD47 expression in the tubules of the kidney. In some embodiments, the glomeruli of the kidney express human CD47 at a level 2 times to 10 times higher than the level of human CD47 expression in the tubules of the kidney. In some embodiments, the level of human CD47 expression is measured by real-time polymerase chain reaction. In some embodiments, the human CD47 is expressed under the same regulatory elements as the endogenous porcine CD47. In some embodiments, the human CD47 is expressed under a glomerulus-specific promoter. In some embodiments, the glomerulus-specific promoter is nephrin. In some embodiments, the kidney is a thymokidney.

### 7. BRIEF DESCRIPTION OF THE DRAWINGS

FIG. 1A-FIG. 1C show phagocytosis of pig endothelial cells (“ECs”) by human macrophages (FIG. 1A), or baboon macrophages (FIG. 1B), and podocytes by baboon macrophages (FIG. 1C).

FIG. 2A-FIG. 2D: show phagocytosis of GalT-KO ECs using human (FIG. 2A), baboon (FIG. 2B), rhesus (FIG. 2C), and cynomolgus (FIG. 2D) macrophages.

FIG. 3A and FIG. 3B: show serum Cre levels after kidney transplantation (FIG. 3A) and histology of a kidney graft (FIG. 3B).

FIG. 4 shows a vector construct for podocyte-specific expression of human CD47. A genomic segment of the porcine nephrin gene (depicted in red) contains the promoter region and additional genomics sequence through the end of the nephrin leader sequence in exon 2. The human CD47 gene (depicted in green) is introduced as a hybrid protein coding sequence/genomic segment comprised of mature protein-coding portions of exons 2-7 (first green region) and the genomic region beginning with intron 7 and continuing through exon 11. This hybrid structure will allow for production of all 4 alternatively spliced isoforms of CD47. A PKG-GFP cassette (depicted in orange) is included for positive selection of transfected fibroblasts that have incorporated the vector into a transcriptionally permissive site.

### 8. DETAILED DESCRIPTION

Provided herein are methods of kidney transplantation from a donor miniature swine expressing human CD47 to a human recipient. Specifically, such donor miniature swine express human CD47 at higher levels in the glomeruli than in the tubules of the kidney. Transgenic donor miniature swine can be generated as described in Section 8.1. Genetic modifications can be introduced in the donor miniature swine using techniques described in Section 8.1. Those donor miniature swine can carry additional genetic modifications (such as alpha-1,3 galactosyltransferase-deficiency) as described in Section 8.1.4. Glomeruli specific expression can be achieved using methods described in Section 8.1.1. Expression levels of human CD47 can be demonstrated using methods described in Section 8.1.3. Transplantation procedures can comprise additional steps, such as bone marrow transplantation, a composite islet-kidney graft, or transplantation of thymic tissue from a miniature swine to the recipient as described in Section 8.2. Immunosuppression and additional conditioning as described in Section 8.2 can be part of the transplantation. As such, the disclosure provides transgenic miniature swine, methods of making thereof, methods of using thereof, with any combination or permutation of the components provided herein. Without being bound by theory, the transplantation methods provided herein result in lower risk and/or severity of renal proteinuria in the transplant recipient.

### 8.1 Generation of Transgenic Miniature Swine

Provided herein are genetically modified swine in which human CD47 is expressed in glomeruli of the kidney at higher levels than in renal tubules of the kidney. The kidneys of such genetically modified swine can be used for transplantation into a human recipient. Without being bound by any particular theory, such an expression pattern of human CD47 in the transplant prevents or reduces proteinuria in the kidney recipient after transplantation. Methods of evaluating proteinuria are provided in section 8.2.4 below.

Glomeruli specific expression of human CD47 can be achieved using methods described in Section 8.1.1. Expression levels of human CD47 can be demonstrated using methods described in Section 8.1.3. Genetic modifications can be introduced in the donor miniature swine using techniques described in Section 8.1. Those donor miniature swine can carry additional genetic modifications (such as alpha-1,3 galactosyltransferase-deficiency) as described in Section 8.1.4.

### 8.1.1. Tissue-Specific Human CD47 Expression

Tissue-specific expression of a human CD47 transgene (e.g., expression of human CD47 in the glomeruli) in a transgenic swine may be achieved by ways of controlling gene expression in a cell type-specific manner. In general, animals can be genetically modified using constructs, which comprise an expression cassette, elements for genomic integration and selection. The expression cassette comprises a promoter and a nucleotide sequence encoding the transgene, e.g., human CD47. Each of these elements is described in detail below. Other methods to achieve tissue-specific expression, such as methods that do not involved genomic integration, can also be used with the methods and compositions provided herein.

In certain embodiments, human CD47 is detectable in endothelial tissues. In certain embodiments, human CD47 is detectable in endothelial tissues of the swine but not in any other tissue. In certain embodiments, human CD47 is detectable in endothelial tissues of the swine but not in the tubules of the kidney of the swine. In certain embodiments, human CD47 is detectable in glomeruli of the kidney of the swine but not detectable in the tubules using a technique described in Section 8.1.3 below. For example, human CD47 may be detectable in one, two or more glomerular cell types. Examples of glomerular cell types include podocytes, glomerular endothelial cells and mesangial cells.

In certain embodiments, human CD47 is detectable only in glomeruli of the kidney of the swine but not detectable in any other tissue of the swine. In other embodiments, human CD47 is detectable in glomeruli of the kidney of the swine and the rest of the body of the swine, but not detectable in the tubules. In certain embodiments, human CD47 is detectable in the bone marrow of a swine and in the glomeruli of the kidney of the swine. In certain embodiments, human CD47 is detectable in the bone marrow of a swine and in the glomeruli of the kidney of the swine, but not in any other tissue of the swine.

In some embodiments, the glomeruli of the kidney of the transgenic swine express human CD47 at a level higher than the level of human CD47 expression in the tubules of the kidney as detected using a technique described in Section 8.1.3 below. In certain embodiments, the glomeruli of the kidney express human CD47 at a level 2 times to 500 times higher than the level of human CD47 expression in the tubules of the kidney. In some embodiments, the glomeruli of the kidney express human CD47 at a level 2 times to 50 times higher than the level of human CD47 expression in the tubules of the kidney. In some embodiments, the glomeruli of the kidney express human CD47 at a level 51 times to 100 times higher than the level of human CD47 expression in the tubules of the kidney. In certain embodiments, the glomeruli of the kidney express human CD47 at a level 101 times to 150 times higher than the level of human CD47 expression in the tubules of the kidney. In some embodiments, the glomeruli of the kidney express human CD47 at a level 151 times to 200 times higher than the level of human CD47 expression in the tubules of the kidney. In some embodiments, the glomeruli of the kidney express human CD47 at a level 201 times to 250 times higher than the level of human CD47 expression in the tubules of the kidney. In certain embodiments, the glomeruli of the kidney express human CD47 at a level 251 times to 300 times higher than the level of human CD47 expression in the tubules of the kidney. In some embodiments, the glomeruli of the kidney express human CD47 at a level 301 times to 350 times higher than the level of human CD47 expression in the tubules of the kidney. In some embodiments, the glomeruli of the kidney express human CD47 at a level 351 times to 400 times higher than the level of human CD47 expression in the tubules of the kidney. In certain embodiments, the glomeruli of the kidney express human CD47 at a level 401 times to 450 times higher than the level of human CD47 expression in the tubules of the kidney. In certain embodiments, the glomeruli of the kidney express human CD47 at a level 451 times to 500 times higher than the level of human CD47 expression in the tubules of the kidney.

In some embodiments, the glomeruli of the kidney express human CD47 at a level at least 2 times, 5 times, 10 times, 25 times, 50 times, 75 times, or at least 100 times higher than the level of human CD47 expression in the tubules of the kidney. In some embodiments, the glomeruli of the kidney express human CD47 at a level at least 2 times higher than the level of human CD47 expression in the tubules of the kidney. In some embodiments, the glomeruli of the kidney express human CD47 at a level at least 5 times higher than the level of human CD47 expression in the tubules of the kidney. In some embodiments, the glomeruli of the kidney express human CD47 at a level at least 10 times higher than the level of human CD47 expression in the tubules of the kidney. In some embodiments, the glomeruli of the kidney express human CD47 at a level at least 25 times higher than the level of human CD47 expression in the tubules of the kidney. In some embodiments, the glomeruli of the kidney express human CD47 at a level at least 50 times higher than the level of human CD47 expression in the tubules of the kidney. In some embodiments, the glomeruli of the kidney express human CD47 at a level at least 75 times higher than the level of human CD47 expression in the tubules of the kidney. In some embodiments, the glomeruli of the kidney express human CD47 at a level at least 100 times higher than the level of human CD47 expression in the tubules of the kidney.

In certain embodiments, the glomeruli of the kidney express human CD47 at a level 2 times to 500 times higher than the level of human CD47 expression in any other tissue in the transgenic swine. In some embodiments, the glomeruli of the kidney express human CD47 at a level 2 times to 50 times higher than the level of human CD47 expression in any other tissue in the transgenic swine. In some embodiments, the glomeruli of the kidney express human CD47 at a level 51 times to 100 times higher than the level of human CD47 expression in any other tissue in the transgenic swine. In certain embodiments, the glomeruli of the kidney express human CD47 at a level 101 times to 150 times higher than the level of human CD47 expression in any other tissue in the transgenic swine. In some embodiments, the glomeruli of the kidney express human CD47 at a level 151 times to 200 times higher than the level of human CD47 expression in any other tissue in the transgenic swine. In some embodiments, the glomeruli of the kidney express human CD47 at a level 201 times to 250 times higher than the level of human CD47 expression in any other tissue in the transgenic swine. In certain embodiments, the glomeruli of the kidney express human CD47 at a level 251 times to 300 times higher than the level of human CD47 expression in any other tissue in the transgenic swine. In some embodiments, the glomeruli of the kidney express human CD47 at a level 301 times to 350 times higher than the level of human CD47 expression in any other tissue in the transgenic swine. In some embodiments, the glomeruli of the kidney express human CD47 at a level 351 times to 400 times higher than the level of human CD47 expression in any other tissue in the transgenic swine. In certain embodiments, the glomeruli of the kidney express human CD47 at a level 401 times to 450 times higher than the level of human CD47 expression in any other tissue in the transgenic swine. In certain embodiments, the glomeruli of the kidney express human CD47 at a level 451 times to 500 times higher than the level of human CD47 expression in any other tissue in the transgenic swine.

In some embodiments, the glomeruli of the kidney express human CD47 at a level at least 2 times, 5 times, 10 times, 25 times, 50 times, 75 times, 100 times higher than the level of human CD47 expression in any other tissue in the transgenic swine. In some embodiments, the glomeruli of the kidney express human CD47 at a level at least 2 times the level of human CD47 expression in any other tissue in the transgenic swine. In some embodiments, the glomeruli of the kidney express human CD47 at a level at least 5 times the level of human CD47 expression in any other tissue in the transgenic swine. In some embodiments, the glomeruli of the kidney express human CD47 at a level at least 10 times the level of human CD47 expression in any other tissue in the transgenic swine. In some embodiments, the glomeruli of the kidney express human CD47 at a level at least 25 times the level of human CD47 expression in any other tissue in the transgenic swine. In some embodiments, the glomeruli of the kidney express human CD47 at a level at least 50 times the level of human CD47 expression in any other tissue in the transgenic swine. In some embodiments, the glomeruli of the kidney express human CD47 at a level at least 75 times the level of human CD47 expression in any other tissue in the transgenic swine. In some embodiments, the glomeruli of the kidney express human CD47 at a level at least 100 times the level of human CD47 expression in any other tissue in the transgenic swine.

In certain embodiments, at least 10%, 20%, 30%, 40%, 50%, at least 55%, at least 60%, at least 65%, at least 70%, at least 75%, at least 80%, at least 85%, at least 90%, or at least 95% of the glomeruli express human CD47. In certain embodiments, at least 10% of the glomeruli express human CD47. In certain embodiments, at least 20% of the glomeruli express human CD47. In certain embodiments, at least 30% of the glomeruli express human CD47. In certain embodiments, at least 40% of the glomeruli express human CD47. In certain embodiments, at least 50% of the glomeruli express human CD47. In certain embodiments, at least 55% of the glomeruli express human CD47. In certain embodiments, at least 60% of the glomeruli express human CD47. In certain embodiments, at least 65% of the glomeruli express human CD47. In certain embodiments, at least 70% of the glomeruli express human CD47. In certain embodiments, at least 75% of the glomeruli express human CD47. In certain embodiments, at least 80% of the glomeruli express human CD47. In certain embodiments, at least 85% of the glomeruli express human CD47. In certain embodiments, at least 90% of the glomeruli express human CD47. In certain embodiments, at least 95% of the glomeruli express human CD47.

In certain embodiments, at least 10%, 20%, 30%, 40%, 50%, at least 55%, at least 60%, at least 65%, at least 70%, at least 75%, at least 80%, at least 85%, at least 90%, or at least 95% of the glomeruli express human CD47 at a higher level than the tubules of the kidney. In certain embodiments, at least 10% of the glomeruli express human CD47 at a higher level than the tubules of the kidney. In certain embodiments, at least 20% of the glomeruli express human CD47 at a higher level than the tubules of the kidney. In certain embodiments, at least 30% of the glomeruli express human CD47 at a higher level than the tubules of the kidney. In certain embodiments, at least 40% of the glomeruli express human CD47 at a higher level than the tubules of the kidney. In certain embodiments, at least 50% of the glomeruli express human CD47 at a higher level than the tubules of the kidney. In certain embodiments, at least 55% of the glomeruli express human CD47 at a higher level than the tubules of the kidney. In certain embodiments, at least 60% of the glomeruli express human CD47 at a higher level than the tubules of the kidney. In certain embodiments, at least 65% of the glomeruli express human CD47 at a higher level than the tubules of the kidney. In certain embodiments, at least 70% of the glomeruli express human CD47 at a higher level than the tubules of the kidney. In certain embodiments, at least 75% of the glomeruli express human CD47 at a higher level than the tubules of the kidney. In certain embodiments, at least 80% of the glomeruli express human CD47 at a higher level than the tubules of the kidney. In certain embodiments, at least 85% of the glomeruli express human CD47 at a higher level than the tubules of the kidney. In certain embodiments, at least 90% of the glomeruli express human CD47 at a higher level than the tubules of the kidney. In certain embodiments, at least 95% of the glomeruli express human CD47 at a higher level than the tubules of the kidney.

In certain embodiments, at least 10%, 20%, 30%, 40%, 50%, at least 55%, at least 60%, at least 65%, at least 70%, at least 75%, at least 80%, at least 85%, at least 90%, or at least 95% of the glomeruli express human CD47 at a higher level than any other tissue of the swine. In certain embodiments, at least 10% of the glomeruli express human CD47 at a higher level than any other tissue of the swine. In certain embodiments, at least 20% of the glomeruli express human CD47 at a higher level than any other tissue of the swine. In certain embodiments, at least 30% of the glomeruli express human CD47 at a higher level than any other tissue of the swine. In certain embodiments, at least 40% of the glomeruli express human CD47 at a higher level than any other tissue of the swine. In certain embodiments, at least 50% of the glomeruli express human CD47 at a higher level than any other tissue of the swine. In certain embodiments, at least 55% of the glomeruli express human CD47 at a higher level than any other tissue of the swine. In certain embodiments, at least 60% of the glomeruli express human CD47 at a higher level than any other tissue of the swine. In certain embodiments, at least 65% of the glomeruli express human CD47 at a higher level than any other tissue of the swine. In certain embodiments, at least 70% of the glomeruli express human CD47 at a higher level than any other tissue of the swine. In certain embodiments, at least 75% of the glomeruli express human CD47 at a higher level than any other tissue of the swine. In certain embodiments, at least 80% of the glomeruli express human CD47 at a higher level than any other tissue of the swine. In certain embodiments, at least 85% of the glomeruli express human CD47 at a higher level than any other tissue of the swine. In certain embodiments, at least 90% of the glomeruli express human CD47 at a higher level than any other tissue of the swine. In certain embodiments, at least 95% of the glomeruli express human CD47 at a higher level than any other tissue of the swine.

In certain embodiments, at least 10%, 20%, 30%, 40%, 50%, at least 55%, at least 60%, at least 65%, at least 70%, at least 75%, at least 80%, at least 85%, at least 90%, or at least 95% of the glomeruli selectively express human CD47. CD47 levels in the glomeruli can be determined using skills known in the art or described herein. In certain embodiments, at least 10% of the glomeruli selectively express human CD47. In certain embodiments, at least 20% of the glomeruli selectively express human CD47. In certain embodiments, at least 30% of the glomeruli selectively express human CD47. In certain embodiments, at least 40% of the glomeruli selectively express human CD47. In certain embodiments, at least 50% of the glomeruli selectively express human CD47. In certain embodiments, at least 55% of the glomeruli selectively express human CD47. In certain embodiments, at least 60% of the glomeruli selectively express human CD47. In certain embodiments, at least 65% of the glomeruli selectively express human CD47. In certain embodiments, at least 70% of the glomeruli selectively express human CD47. In certain embodiments, at least 75% of the glomeruli selectively express human CD47. In certain embodiments, at least 80% of the glomeruli selectively express human CD47. In certain embodiments, at least 85% of the glomeruli selectively express human CD47. In certain embodiments, at least 90% of the glomeruli selectively express human CD47. In certain embodiments, at least 95% of the glomeruli selectively express human CD47.

In certain embodiments, both the glomeruli of the kidney and the bone marrow of a transgenic swine express human CD47. In some embodiments, the glomeruli of the kidney and the bone marrow of the human swine are the only two tissues wherein expression of human CD47 is detectable, for example, detectable by a method described in section 8.1.3 below.

Any method known to the skilled artisan can be used to quantify levels of human CD47 (e.g., human CD47 gene or protein expression levels). In certain embodiments, the human CD47 expression level in the glomeruli, as detected using a technique described in Section 8.1.3 below, are normalized using expression level of one or more housekeeping genes in the glomeruli. In other embodiments, the human CD47 expression level in the glomeruli are normalized using historical expression levels of one or more housekeeping genes in the glomeruli. In certain embodiments, the human CD47 expression level in the tubules, as detected using a technique described in Section 8.1.3 below, are normalized using expression level of one or more housekeeping gene in the tubules. In other embodiments, the human CD47 expression level in the tubules are normalized using historical expression levels of one or more housekeeping genes in the tubules. Housekeeping genes are well-known in the art and include, for example, β-actin, glyceraldehyde 3-phosphate dehydrogenase (GAPDH) or histone.

In some embodiments, all glomeruli present in the kidney express human CD47 at a level higher than the level of human CD47 expression in the tubules. In some embodiments, more than 75% of the glomeruli of the kidney express human CD47 at a level higher than the level of human CD47 expression in the tubules. In some embodiments, more than 50% of the glomeruli of the kidney express human CD47 at a level higher than the level of human CD47 expression in the tubules. In some embodiments, more than 25% of the glomeruli of the kidney express human CD47 at a level higher than the level of human CD47 expression in the tubules.

### 8.1.2. Constructs For The Generation Of Transgenic Donor Animals

Methods of making transgenic animals (e.g., miniature swine) are well known in the art. See, e.g., Hryhorowicz et al. (2020), Genes 2020, 11, 670. Examples of such methods are described herein below. In certain embodiments, miniature swine from an inbred herd of miniature swine are used. The transgenic animal may be produced by any suitable method known in the art. Thus, the gene expression construct (for example, a construct described herein) may be introduced into the germline of the animal using, for example, somatic cell nuclear transfer (SCNT), pronuclear microinjection, sperm-mediated gene transfer (SMGT) or viral-mediated transgenesis See, e.g., Yum et al. (2016) J Vet Sci 2016, 17:261-268; Whyte and Prather (2011), Mol Reprod Dev 78:879-891; Sachs and Gali (2009).

SCNT involves the transfer of the nucleus of a donor cell into an oocyte or early embryo from which the chromosomes have been removed. See, e.g., Wilmut and Taylor (2015), Phil. Trans. R. Soc. B 370:20140366. Pronuclear microinjection involves the direct injection of DNA into the pronuclei. Eggs for these purposes may be collected from a superovulated females, and then transferred to a recipient pig by embryo transfer. See, e.g., Whyte and Prather (2011), Mol Reprod Dev 78:879-891. SMGT involves incubating genes for the transgene of interest with spermatozoa which are subsequently used for insemination. See, e.g., Lavitrano et al., (2002), Proc Nat Acad Sci USA. 99:14230-14235. Viral-mediated Transgenesis relies on infection of an embryo or oocyte with a viral vector carrying the transgene. Exemplary viral vectors include adeno-associated virus (AAV), self-complimentary adeno-associated virus (scAAV), adenovirus, retrovirus, lentivirus (e.g., Simian immunodeficiency virus, human immunodeficiency virus, or modified human immunodeficiency virus), Newcastle disease virus (NDV), herpes virus (e.g., herpes simplex virus), alphavirus, vaccina virus, etc.).

Constructs for the expression of transgenes generally comprise elements for genomic integration and selection, as well as an expression cassette. The expression cassette comprises a promoter and a nucleotide sequence encoding the transgene, e.g., human CD47. Viral vectors may further comprise other elements, such as a Poly(A) site, a transcription termination site, or viral-specific elements such as inverted terminal repeats. See, e.g. Buard et al. (2009), British Journal of Pharmacology 157:153-165.

### 8.1.2.1 Elements for Genomic Integration

The sequence-specific insertion (or knock-in) of human CD47 transgene into the genome of the donor miniature swine may be achieved by a sequence-specific endonuclease coupled with homologous recombination (HR) of the targeted chromosomal locus with the construct containing the transgene of human CD47. See, e.g., Meyer et al. (2010), Proc. Natl. Acad. Sci. USA 107:15022-15026; Cui et al. (2010), Nat. Biotechnol. 29:64-67; Moehle et al. (2007), Proc Natl Acad Sci USA 104:3055-3060. This process relies on targeting specific gene sequences with endonucleases that recognize and bind to such sequences and induce a double-strand break in the nucleic acid molecule of the miniature swine cell. The double-strand break is then repaired by homologous recombination. If a template (e.g., a construct containing the human CD47) for homologous recombination is provided in trans, the double-strand break can be repaired using the provided template. Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas9).

Another example of sequence-specific endonucleases includes RNA-guided DNA nucleases, e.g., the CRISPR/Cas system. The Cas9/CRISPR (Clustered Regularly-Interspaced Short Palindromic Repeats) system exploits RNA-guided DNA-binding and sequence-specific cleavage of target DNA. A guide RNA (gRNA) (e.g., containing 20 nucleotides) are complementary to a target genomic DNA sequence upstream of a genomic PAM (protospacer adjacent motifs) site (NNG) and a constant RNA scaffold region. The Cas (CRISPR-associated) protein binds to the gRNA and the target DNA to which the gRNA binds and introduces a double-strand break in a defined location upstream of the PAM site. See, e.g., Geurts et al. (2009), Science 325:433; Mashimo et al. (2010), PLoS ONE 5, e8870; Carbery et al. (2010), Genetics 186:451-459; Tesson et al. (2011), Nat. Biotech. 29:695-696; Wiedenheft et al. (2012), Nature 482,331-338; Jinek et al. (2012), Science 337:816-821; Mali et al. (2013), Science 339:823-826; Cong et al. (2013), Science 339:819-823.

In one embodiment, a sequence-specific recombination system may be used to achieve the conditional knockout of the target gene (e.g. swine CD47). The recombinase is an enzyme that recognizes specific polynucleotide sequences (recombinase recognition sites) that flank an intervening polynucleotide and catalyzes a reciprocal strand exchange, resulting in inversion or excision of the intervening polynucleotide. See, e.g., Araki et al. (1995), Proc. Natl. Acad. Sci. USA 92:160-164.

In another aspect, a transgene may be integrated in a sequence non-specific way using, e.g., non-homologous end joining.

In another embodiment, conditional expression of the transgene (which encodes, e.g., a recombinase, or human CD47 transgene) can be achieved by using regulatory sequence that can be induced or inactivated by exogenous stimuli. For example, the sequence-specific recombination system of the conditional knock-out allele can be regulated, by, e.g., having the activity of the recombinase to be inducible by a chemical (drug). The chemical may activate the transcription of the Cre recombinase gene, or activates transport of the Cre recombinase protein to the nucleus. Alternatively, the recombinase can be activated by the absence of an administered drug rather than by its presence. Non-limiting examples of the chemicals regulating the inducible system (thus, e.g., inducing conditional knockouts) include tetracycline, tamoxifen, RU-486, doxycycline, and the like. See, e.g., Nagy A (2000), Genesis, 26: 99-109. See, for example, the conditional knock-out and knock-in construct described in U.S. patent application Ser. No. 15/558,789.

In other embodiments, the endogenous porcine CD47 is replaced with human CD47 at the endogenous locus (i.e., gene knock-in). Various techniques known in the art can be used to generate human CD47 knock-in models. For example, one non-limiting example includes using a combination of CRISPR/Cas9 and somatic cell nuclear transfer. See, e.g., Ruan J, et al. Sci Rep. 2015 Sep. 18; 5:14253.

### 8.1.2.2 Expression Cassettes

Expression cassettes generally comprise a regulatory element and a transgene. A regulatory element may be, for example, a promoter. Thus, for example, to achieve expression of a human CD47 transgene in the glomeruli, the transgene is placed under the control of a glomerulus-specific promoter (see section 8.1.2.4).

### 8.1.2.3 Transgenes

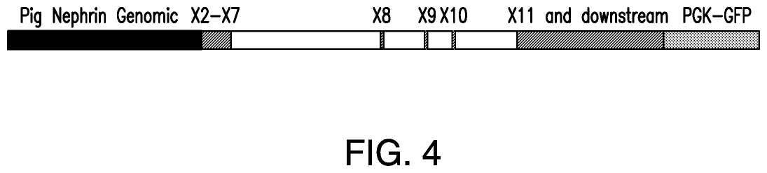

Amino acid sequences of human CD47 can be found under the following NCBI Reference Sequence (RefSeq) accession numbers: NP_001768; NP_001369235.1; NP_942088; and XP_005247966.1. Nucleic acid sequences encoding human CD47 can be found under the following NCBI RefSeq accession numbers: NM_001777; NM_198793; XM_005247909.2 and NM_001382306.1. In some embodiments, a transgene provided herein encodes a known splice variant of human CD47. In some embodiments, a transgene provided herein is a hybrid of cDNA and genomic DNA forms that provides for the production of multiple splice forms from a single transgenic construct (FIG. 4).

Sequences of CD47 in other species are also known. See, for example, the amino acid sequences under the following NCBI RefSeq numbers: XP 516636 (chimpanzee); and XP 535729 (dog); Polypeptides which include all or a portion of the extracellular domain of CD47 are contemplated herein. See, e.g., Motegi et al. (2003), EMBO J., 22: 2634-2644, which describes the construction of a human CD47-Fc fusion protein. In some embodiments provided herein, alternatively spliced forms of human CD47 are used. See, e.g., Reinhold et al. (1999), Journal of Cell Science, 108:3419-3425. In certain embodiments, the transgene encoding human CD47 used in a construct described herein is a transgene listed in Table 1 below. In certain embodiments, the transgene encoding human CD47 comprises a nucleotide sequence of SEQ ID NO: 3. In other embodiments, the transgene encoding human CD47 comprises a nucleotide sequence of SEQ ID NO: 4.

In certain embodiments, the transgene encoding human CD47 comprises a nucleotide sequence that is at least 70%, at least 75%, at least 80%, at least 85%, at least 90%, at least 95% or at least 98% identical to SEQ ID NO: 3. In certain embodiments, the transgene encoding human CD47 comprises a nucleotide sequence that is at least 70% identical to SEQ ID NO: 3. In certain embodiments, the transgene encoding human CD47 comprises a nucleotide sequence that is at least 75% identical to SEQ ID NO: 3. In certain embodiments, the transgene encoding human CD47 comprises a nucleotide sequence that is at least 80% identical to SEQ ID NO: 3. In certain embodiments, the transgene encoding human CD47 comprises a nucleotide sequence that is at least 85% identical to SEQ ID NO: 3. In certain embodiments, the transgene encoding human CD47 comprises a nucleotide sequence that is at least 90% identical to SEQ ID NO: 3. In certain embodiments, the transgene encoding human CD47 comprises a nucleotide sequence that is at least 95% identical to SEQ ID NO: 3. In certain embodiments, the transgene encoding human CD47 comprises a nucleotide sequence that is at least 98% identical to SEQ ID NO: 3.

In other embodiments, the transgene encoding human CD47 comprises a nucleotide sequence that is at least 70%, at least 75%, at least 80%, at least 85%, at least 90%, at least 95% or at least 98% identical to SEQ ID NO: 4. In certain embodiments, the transgene encoding human CD47 comprises a nucleotide sequence that is at least 70% identical to SEQ ID NO: 4. In certain embodiments, the transgene encoding human CD47 comprises a nucleotide sequence that is at least 75% identical to SEQ ID NO: 4. In certain embodiments, the transgene encoding human CD47 comprises a nucleotide sequence that is at least 80% identical to SEQ ID NO: 4. In certain embodiments, the transgene encoding human CD47 comprises a nucleotide sequence that is at least 85% identical to SEQ ID NO: 4. In certain embodiments, the transgene encoding human CD47 comprises a nucleotide sequence that is at least 90% identical to SEQ ID NO: 4. In certain embodiments, the transgene encoding human CD47 comprises a nucleotide sequence that is at least 95% identical to SEQ ID NO: 4. In certain embodiments, the transgene encoding human CD47 comprises a nucleotide sequence that is at least 98% identical to SEQ ID NO: 4.

In certain embodiments, the transgene encodes a polypeptide of SEQ ID NO: 1. In certain embodiments, the transgene encodes a polypeptide that is at least 70%, at least 75%, at least 80%, at least 85%, at least 90%, at least 95% or at least 98% identical to SEQ ID NO: 1. In certain embodiments, the transgene encoding human CD47 comprises a nucleotide sequence that is at least 70% identical to SEQ ID NO: 1. In certain embodiments, the transgene encoding human CD47 comprises a nucleotide sequence that is at least 75% identical to SEQ ID NO: 1. In certain embodiments, the transgene encoding human CD47 comprises a nucleotide sequence that is at least 80% identical to SEQ ID NO: 1. In certain embodiments, the transgene encoding human CD47 comprises a nucleotide sequence that is at least 85% identical to SEQ ID NO: 1. In certain embodiments, the transgene encoding human CD47 comprises a nucleotide sequence that is at least 90% identical to SEQ ID NO: 1. In certain embodiments, the transgene encoding human CD47 comprises a nucleotide sequence that is at least 95% identical to SEQ ID NO: 1. In certain embodiments, the transgene encoding human CD47 comprises a nucleotide sequence that is at least 98% identical to SEQ ID NO: 1.

In other embodiments, the transgene encodes a polypeptide of SEQ ID NO: 2. In certain embodiments, the transgene encodes a polypeptide that is at least 70%, at least 75%, at least 80%, at least 85%, at least 90%, at least 95% or at least 98% identical to SEQ ID NO: 2. In certain embodiments, the transgene encoding human CD47 comprises a nucleotide sequence that is at least 70% identical to SEQ ID NO: 2. In certain embodiments, the transgene encoding human CD47 comprises a nucleotide sequence that is at least 75% identical to SEQ ID NO: 2. In certain embodiments, the transgene encoding human CD47 comprises a nucleotide sequence that is at least 80% identical to SEQ ID NO: 2. In certain embodiments, the transgene encoding human CD47 comprises a nucleotide sequence that is at least 85% identical to SEQ ID NO: 2. In certain embodiments, the transgene encoding human CD47 comprises a nucleotide sequence that is at least 90% identical to SEQ ID NO: 2. In certain embodiments, the transgene encoding human CD47 comprises a nucleotide sequence that is at least 95% identical to SEQ ID NO: 2. In certain embodiments, the transgene encoding human CD47 comprises a nucleotide sequence that is at least 98% identical to SEQ ID NO: 2.

In one embodiments, the human CD47 transgene is inserted into a locus other than the natural locus of the swine CD47 gene in the transgenic donor animal.

### 8.1.2.4 Regulatory Elements

In certain embodiments, the human CD47 transgene is under the control of a glomerulus-specific promoter. In some embodiments, the glomerulus-specific promoter is specific to one or more glomerular cell types. Examples of glomerular cell types include podocytes, mesangial cells and glomerular endothelial cells. In certain embodiments, the glomerulus-specific promoter is a podocyte-specific promoter. In certain embodiments, the glomerulus-specific promoter is the nephrin promoter. In certain embodiments, the glomerulus-specific promoter is the podocin promoter. In certain embodiments, the glomerulus-specific promoter is the FGF1 promoter. In certain embodiments, the glomerulus-specific promoter is a mesangial cell-specific promoter. In certain embodiments, the glomerulus-specific promoter is an endothelial cell-specific promoter. In certain embodiments, the glomerulus-specific promoter is the CD31 promoter. In certain embodiments, the glomerulus-specific promoter is the vWF promoter.

A promoter may control gene expression in more than one cell type. In certain embodiments, the promoter controls gene expression in glomerular cells. In certain embodiments, the promoter controls gene expression in glomerular cell type podocytes.

In certain embodiments, the promoter of any gene expressed in glomeruli can be analyzed and the regulatory elements that confer expression in glomeruli can be used with the methods and compositions provided herein. In general, such a promoter analysis can be conducted by recombinantly placing a reporter gene (such as a fluorescent protein) under the regulatory control of fragments of the gene of interest. The resulting construct can then be tested for expression of the reporter gene in glomeruli.

In certain embodiments, a promoter may be inducible. Specifically a promoter may be inducible and tissue-specific. Numerous inducible promoters and gene expression systems are known in the art. For example, a promoter may be induced by a chemical, e.g., by tetracyclin, tamoxifen, or cumate. Gene expression can also be controlled by protein-protein interactions (e.g., the interaction between FKBP12 and mTOR, which is controlled by rapamycin). See, e.g., Kallunki et al. (2019), Cells 8:796.

### 8.1.3. Methods of Measuring Human CD47 Levels

In certain embodiments, levels of human CD47 expression can be determined at the RNA (e.g., mRNA level) as in Section 8.1.3.1 discussed below. In certain embodiments, levels of human CD47 expression can be determined at the protein level as discussed in Section 8.1.3.2.

In certain embodiments, the methods provided herein include methods of detecting and measuring differential gene expression in kidney glomeruli tissue versus the kidney tubuli tissue of the donor miniature swine. In certain embodiments, the methods provided herein include methods of detecting and measuring differential mRNA levels of human CD47 in kidney glomeruli tissue versus kidney tubuli tissue of the donor miniature swine. In other embodiments, the methods provided herein include methods of detecting and measuring differential protein levels of human CD47 in kidney glomeruli tissue versus the kidney tubuli tissue of the donor miniature swine.

Tissue-specific expression may be determined by physically isolating the tissue of interest before measuring human CD47 protein or mRNA levels (e.g., by renal biopsy or by flow cytometry-based isolation of e.g., glomeruli-specific cells) and applying methods to measure human CD47 protein or mRNA levels such as the ones below in vitro. Alternatively, imagining techniques such as fluorescent microscopy may be used to visualize and measure human CD47 protein expression in specific tissues (e.g., the glomeruli or the tubules). Single cell qPCR may be used to measure human CD47 gene expression in specific tissues.

In some embodiments, the methods provided herein include (i) performing renal biopsy of the donor miniature swine; (ii) isolating glomeruli from the kidneys of the donor miniature swine; and/or (iii) isolating tubules from the kidneys of the donor miniature swine. In other embodiments, the methods provided herein include (i) performing renal biopsy of the donor miniature swine; and (ii) dissecting the kidneys of the donor miniature swine to level of individual or group of nephrons. In some embodiments, the above methods are performed in combination.

### 8.1.3.1 Methods of Detecting mRNA Levels in a Sample

In certain embodiments, mRNA of human CD47 is detected in glomeruli of the kidney of the swine but not detected in the tubules by a technique described herein. In some embodiments, the glomeruli of the kidney have higher level of mRNA of human CD47 than the mRNA level of human CD47 in the tubules of the kidney as detected using a technique described herein.

Several methods of detecting or quantifying mRNA levels are known in the art. Exemplary methods include, but are not limited to, northern blots, ribonuclease protection assays, PCR-based methods (e.g., quantitative PCR), RNA sequencing, Fluidigm® analysis, and the like. The mRNA sequence of a human CD47 can be used to prepare a probe that is at least partially complementary to the mRNA sequence. The probe can then be used to detect the mRNA in a sample, using any suitable assay, such as PCR-based methods, northern blotting, a dipstick assay, TaqMan™ assays and the like.

In other embodiments, a nucleic acid assay for testing for human CD47 expression in a biological sample can be prepared. An assay typically contains a solid support and at least one nucleic acid contacting the support, where the nucleic acid corresponds to at least a portion of the mRNA. The assay can also have a means for detecting the altered expression of the mRNA in the sample. The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

A typical mRNA assay method can contain the steps of: (1) obtaining surface-bound subject probes; (2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Other methods, such as PCR-based methods, can also be used to detect the expression of human CD47. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). In some embodiments, qRT-PCR-based assays can be useful to measure mRNA levels during cell-based assays. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT-PCR gives quantitative results. An additional advantage of qRT-PCR is the relative ease and convenience of use. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. An exemplary qRT-PCR program, for example, is 50° C. for 2 minutes, 95° C. for 10 minutes, 40 cycles of 95° C. for 15 seconds, then 60° C. for 1 minute.

### 8.1.3.2 Methods of Detecting Polypeptide or Protein Levels in a Sample

In certain embodiments provided herein, human CD47 polypeptide or protein is detected in glomeruli of the kidney of the swine but not detected in the tubules by a technique described herein. In some embodiments, the glomeruli of the kidney have higher level of human CD47 polypeptide or protein than the level of human CD47 polypeptide or protein in the tubules of the kidney as detected using a technique described herein.

Several protein detection and quantification methods can be used to measure the level of human CD47. Any suitable protein quantification method can be used. In some embodiments, antibody-based methods are used. Exemplary methods that can be used include, but are not limited to, immunoblotting (Western blot), ELISA, immunohistochemistry, immunofluorescence, flow cytometry, cytometry bead array, mass spectroscopy, and the like. Several types of ELISA are commonly used, including direct ELISA, indirect ELISA, and sandwich ELISA.

### 8.1.4. Other Genetic Modifications

Recombinant miniature swine provided herein (for example, the first miniature swine and/or the second miniature used in a method of transplantation described herein) may be modified in additional ways to the expression of human CD47. Such additional modifications include, for example, knockout of α-1,3-galactosyltransferase and modifications of the cytokine receptors. In some embodiment, a miniature swine provided herein does not express α-1,3-galactosyltransferase. In some embodiments, a miniature swine provided herein additionally expresses human CD55, human CD46, human CD59, IL-3R, or some combination thereof. See, e.g., Nomura et al. (2020), Xenotransplantation. 2020; 27:e12549, U.S. Pat. Nos. 9,883,939 and 9,980,471 B2.

Referring to the transplantation methods in Section 8.2, such additional genetic modifications can be used in connection with the miniature swine that is the donor for a kidney transplant, and such additional modifications can also be used in connection with the miniature swine that is the donor for hematopoietic stem cells (e.g., for a bone marrow transplant).

### 8.2 Methods of Transplantation

Cells, tissues, organs or body fluids of the transgenic donor miniature swine may be used in methods of transplantation (e.g., xenotransplantation).

Recipients may be transplanted with a first and a second graft from one or two animals. In some embodiments, the second graft harvested from the donor animal is transplanted at least 7 days after transplantation of first graft from the donor animal. In some embodiments, the second graft harvested from the donor animal is transplanted at least 14 days after transplantation of first graft from the donor animal. In some embodiments, the second graft harvested from the donor animal is transplanted at least 21 days after transplantation of first graft from the donor animal. In some embodiments, the second graft harvested from the donor animal is transplanted at least 28 days after transplantation of first graft from the donor animal. In some embodiments, the second graft harvested from the donor animal is transplanted at least 35 days after transplantation of first graft from the donor animal. In some embodiments, the second graft harvested from the donor animal is transplanted at least 49 days after transplantation of first graft from the donor animal. In some embodiments, the second graft harvested from the donor animal is transplanted at least 54 days after transplantation of first graft from the donor animal.

In one embodiment, a method of transplantation provided herein comprises transplantation of a kidney with the genetic modification described in Section 8.1 from a donor animal. In certain aspects, the methods of transplantation provided herein comprise steps to induce tolerance in the recipient, e.g., by inducing mixed chimerism. “Mixed chimerism” is commonly understood to describe a state in which the lymphohematopoietic system of the recipient of allogeneic hematopoietic stem cells comprises a mixture of host and donor cells. This state is usually attained through either bone marrow or mobilized peripheral blood stem cell transplantation. Mixed chimerism may be transient or stable. See, e.g., Sachs et al. (2014), Cold Spring Harb Perspect Med 2014;4:a015529; U.S. Pat. Nos. 6,296,846 and 6,306,651. Mixed chimerism may also be achieved by concurrent transplantation of thymic tissue from the donor animal. See, e.g., International Patent Application Publication No. WO2020/061272.

In one embodiment, the present disclosure includes a method of transplanting a kidney from a second donor animal into a human recipient, wherein the method comprises: (a) transplanting hematopoietic stem cells from a first donor animal to the recipient; and (b) transplanting a kidney from a second donor animal to the recipient, wherein the first donor animal expresses human CD47 in the hematopoietic stem cells and the second donor animal selectively expresses human CD47 in the glomeruli of the kidney. In a specific embodiment, the first donor animal is a miniature swine. In a specific embodiment, the second donor animal is a miniature swine. In a specific embodiment, both the first and the second donor animals are miniature swine. In other specific embodiments, the second donor animal is a miniature swine and the first donor animal is not a miniature swine. In some embodiments, the method of transplantation optionally include the transplantation of thymic tissue from a third donor animal.

In one embodiment, the present disclosure includes a method of transplanting a kidney from a second donor animal into a human recipient, wherein the method comprises: (a) transplanting hematopoietic stem cells and thymic tissue from a first donor animal to the recipient; and (b) transplanting a kidney from a second donor animal to the recipient, wherein the first donor animal expresses human CD47 in the hematopoietic stem cells, and the second donor animal selectively expresses human CD47 in the glomeruli of the kidney. In a specific embodiment, the first donor animal is a miniature swine. In a specific embodiment, the second donor animal is a miniature swine. In a specific embodiment, both the first and the second donor animals are miniature swine. In other specific embodiments, the second donor animal is a miniature swine and the first donor animal is not a miniature swine. In some embodiments, the thymic tissue from the first donor animal expresses human CD47. Examples of thymic tissue include vascularized thymic tissue and thymokidneys (see section 8.2.1.2),In one embodiment, the present disclosure includes a method of transplanting a kidney from a miniature swine into a human recipient, wherein the method comprises: (a) transplanting hematopoietic stem cells from a first miniature swine to the recipient; and (b) transplanting a kidney from a second miniature swine to the recipient, wherein the first swine expresses human CD47 in the hematopoietic stem cells and the second swine selectively expresses human CD47 in the glomeruli of the kidney. The first swine may also express human CD47 in tissues other than the hematopoietic stem cells.

In certain embodiments of the method, said second step of transplanting a kidney from a second miniature swine is carried out at least 28 days after first step of transplanting hematopoietic stem cells from a first miniature swine. The present disclosure includes the methods and techniques described in Watanabe et al., Xenotransplantation, 2020, 27:e12552 and Nomura et al., Xenotransplantation, 2020, 27:e12549 for transgenic expression of human CD47 in donor cells.

The hematopoietic stem cells can be any type of cell. In certain embodiments, the cell is a hematopoietic stem cell, lymphocyte, or a myeloid cell. In some embodiments, a mixed population of hematopoietic cells is transplanted from the first donor animal (e.g., miniature swine) into the recipient. In certain embodiments, the porcine hematopoietic stem cells are obtained from bone marrow, peripheral blood, umbilical cord blood, fetal liver or embryonic stem cells. The hematopoietic stem cells may be transplanted by any suitable method known in the art, for example by a method described in section 8.2.1.3 below. In some embodiments, the hematopoietic stem cells are transplanted to the recipient by intra bone-bone marrow transplantation, e.g. as described in Watanabe et al. (2019), Xenotransplantation. 2019; 00:e12552.

In some embodiments, the hematopoietic stem cells and the donor kidney are taken from the same donor animal. In some embodiments wherein the hematopoietic stem cells and the kidney are taken from the same donor, the donor hematopoietic stem cells and the glomeruli of the donor kidney express human CD47. In some embodiments wherein the hematopoietic stem cells and the kidney are taken from the same donor, the donor hematopoietic stem cells and the glomeruli of the donor kidney express human CD47 at higher levels than the kidney tubules. In some embodiments wherein the hematopoietic stem cells and the kidney are taken from the same donor, the donor hematopoietic stem cells and the glomeruli of the donor kidney express human CD47 at higher levels than any other tissue in the donor animal.

In some embodiments, the hematopoietic stem cells and the donor kidney are taken from two different, but genetically matched donor animals. “Genetically matched” as used herein may refer to homology between genes, for example, MHC genes. In some embodiments, the genetically matched donor animals are perfectly matched for MHC. In some embodiments, the hematopoietic stem cells and the donor kidney are taken from two different animals from the same, highly inbred herd.

### 8.2.1. Additional Treatments

Additional treatments may be used prior to, concurrently with, or subsequent to the methods of transplantation described herein. Additional treatments are generally intended to improve the tolerance of the xenograft in the recipient, but other treatments are contemplated. A method of transplantation provided herein can thus include administering one or more additional treatments, e.g., a treatment which inhibits T cells, blocks complement, or otherwise down regulates the recipient immune response to the graft.

In some embodiments, a recipient is thymectomized and/or splenectomized.

In some embodiments, a recipient receives radiation, for example, total body irradiation. In specific embodiments, a recipient receives 5-10 Gy or 10-15 Gy irradiation. In some embodiments, thymic irradiation can be used. In some embodiments, the recipient is administered low dose radiation (e.g., a sub lethal dose of between 100 rads and 400 rads whole body radiation). Local thymic radiation may also be used.

The blood of a subject undergoing transplantation by a method described herein may contain antibodies that target the xenograft. Such antibodies can be eliminated by organ perfusion, and/or transplantation of tolerance-inducing bone marrow. Natural antibodies can be absorbed from the recipient's blood by hemoperfusion of a liver of the donor species. Similarly, antibody-producing cells may be present in the recipient. Such antibody producing cells may be eliminated by, for example, irradiation or drug treatments In certain embodiment, the graft, cells, tissues, or organs used for transplantation may be genetically modified such that they are not recognized by antibodies present in the host (e.g., the cells are a-1,3-galactosyltransferase deficient) per Section 8.1.4.

In some embodiments, donor stromal tissue is administered. It may be obtained from fetal liver, thymus, and/or fetal spleen, may be implanted into the recipient, e.g., in the kidney capsule.

### 8.2.1.1 Immunosuppressive Therapy

In some embodiments, the patient receiving a xenograft in accordance with the methods described herein receives immunosuppressive therapy. The immunosuppressive therapy may be any FDA-approved treatment indicated to reduce transplant rejection and/or ameliorate the outcome of xenotransplantation. Non-limiting examples of immunosuppressive therapy include calcineurin inhibitors (e.g., tacrolimus or cyclosporine), antiproliferative agents (e.g., anti-metabolites such a mycophenolate, 6-mercaptopurine or its prodrug azathioprine), inhibitors of mammalian target of rapamycin (mTOR) (e.g., sirolimus, rapamycin), steroids (e.g., prednisone), cell cycle inhibitors (azathioprine or mycophenolate mofetil), lymphocyte-depleting agents (e.g., anti-thymocyte globulin or antibodies such as alemtuzumab, siplizumab or basiliximab) and co-stimulation blockers (e.g., belatacept). See, e.g., Chung et al (2020)., Ann Transl Med. March; 8(6): 409; van der Mark et al. (2020), Eur Respir Rev; 29: 190132 and Benvenuto et al. (2018), J Thorac Dis 10:3141-3155. In some embodiments, the immunosuppressive therapy includes a calcineurin inhibitor. In some embodiments, the immunosuppressive therapy includes an antiproliferation agent. In some embodiments, the immunosuppressive therapy includes an inhibitor of mTOR. In some embodiments, the immunosuppressive therapy includes a steroid. In some embodiments, the immunosuppressive therapy includes a lymphocyte-depleting agent. In some embodiments, the immunosuppressive therapy includes a co-stimulation blocker.

Immunosuppressive therapy may be administered as induction therapy (perioperative, or immediately after surgery) a maintenance dose or for an acute rejection. Induction therapy commonly includes basiliximab, anti-thymocyte globulin or alemtuzumab. Immunosuppressive therapy may also be administered as maintenance therapy which is often required to continue for the life of the recipient. Maintenance immunosuppressive therapy commonly includes a calcineurin inhibitor (tacrolimus or cyclosporine), an antiproliferative agent (mycophenolate or azathioprine), and corticosteroids. Immunosuppressive therapy for acute rejections commonly includes thymoglobulin or mycophenolate. See, e.g., Chung et al. (2020), Ann Transl Med. Mar; 8: 409 and Benvenuto et al., (2018) J Thorac Dis 10:3141-3155.

Non-limiting examples of immunosuppressants include, (1) antimetabolites, such as purine synthesis inhibitors (such as inosine monophosphate dehydrogenase (IMPDH) inhibitors, e.g., azathioprine, mycophenolate, and mycophenolate mofetil), pyrimidine synthesis inhibitors (e.g., leflunomide and teriflunomide), and antifolates (e.g., methotrexate); (2) calcineurin inhibitors, such as tacrolimus, cyclosporine A, pimecrolimus, and voclosporin; (3) TNF-alpha inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus, and biolimus A9; (6) corticosteroids, such as prednisone; and (7) antibodies to any one of a number of cellular or serum targets (including anti-lymphocyte globulin and anti-thymocyte globulin).

Non-limiting exemplary cellular targets and their respective inhibitor compounds include, but are not limited to, complement component 5 (e.g., eculizumab); tumor necrosis factors (TNFs) (e.g., infliximab, adalimumab, certolizumab pegol, afelimomab and golimumab); IL-5 (e.g., mepolizumab); IgE (e.g., omalizumab); BAYX (e.g., nerelimomab); interferon (e.g., faralimomab); IL-6 (e.g., elsilimomab); IL-12 and IL-13 (e.g., lebrikizumab and ustekinumab); CD3 (e.g., muromonab-CD3, otelixizumab, teplizumab, visilizumab); CD4 (e.g., clenoliximab, keliximab and zanolimumab); CDI la (e.g., efalizumab); CD18 (e.g., erlizumab); CD20 (e.g., afutuzumab, ocrelizumab, pascolizumab); CD23 (e.g., lumiliximab); CD40 (e.g., teneliximab, toralizumab); CD62L/L-selectin (e.g., aselizumab); CD80 (e.g., galiximab); CD147/basigin (e.g., gavilimomab); CD154 (e.g., ruplizumab); BLyS (e.g., belimumab); CTLA-4 (e.g., ipilimumab, tremelimumab); CAT (e.g., bertilimumab, lerdelimumab, metelimumab); integrin (e.g., natalizumab); IL-6 receptor (e.g., tocilizumab); LFA-1 (e.g., odulimomab); and IL-2 receptor/CD25 (e.g., basiliximab, daclizumab, inolimomab).

### 8.2.1.2 Vascularized Thymic Transplant

In some embodiments, a patient treated in accordance with a method described here receives a vascularized thymic transplant. See, e.g., International Patent Application Publication No. PCT WO2020061272A1. Thymic tissue can be prepared for transplantation by implantation under the autologous kidney capsule for revascularization. A vascularized thymic transplant can be, for example, a “thymokidney,” i.e., a kidney prepared by transplanting thymic tissue from a donor under the donor's own kidney capsule. See, e.g., Yamada et. al., Transplantation 68(11):1684-1692 (1999), Yamada et al., J Immunol 164:3079-3086 (2000) and Yamada et al., Transplantation 76(3):530-536 (2003). A vascularized thymic transplant can also be a vascularized thymic lobe transplanted separately from the kidney. See, e.g., LaMattina et al., Transplantation 73(5):826-831 (200) and Kamano et al., Proc Natl Acad Sci U S A 101(11):3827-3832 (2004).

### 8.2.1.3 Hematopoietic Stem Cell Transplant

Stem cell engraftment and hematopoiesis across disparate species barriers may be enhanced by providing a hematopoietic stromal environment from the donor species. The stromal matrix supplies species-specific factors that are required for interactions between hematopoietic stem cells and their stromal environment, such as hematopoietic growth factors, adhesion molecules, and their ligands.

As liver is the major site of hematopoiesis in the fetus, fetal liver can also serve as an alternative to bone marrow as a source of hematopoietic stem cells. The thymus is the major site of T cell maturation. Each organ includes an organ specific stromal matrix that can support differentiation of the respective undifferentiated stem cells implanted into the host. Thymic stromal tissue can be irradiated prior to transplantation. As an alternative or an adjunct to implantation, fetal liver cells can be administered in fluid suspension.

Bone marrow cells (BMC), or another source of hematopoietic stem cells, e.g., a fetal liver suspension, of the donor can be injected into the recipient in order to induce mixed chimerism. The hematopoietic stem cells may be taken from any source, for example from the bone marrow or peripheral blood stem cells. See, e.g., Sachs et al. (2014), Cold Spring Harb Perspect Med 2014; 4:a015529. Donor BMC home to appropriate sites of the recipient and grow contiguously with remaining host cells and proliferate, forming a chimeric lymphohematopoietic population. By this process, newly forming B cells (and the antibodies they produce) are exposed to donor antigens, so that the transplant will be recognized as self. Tolerance to the donor is also observed at the T cell level in animals in which hematopoietic stem cell, e.g., bone marrow cell, engraftment has been achieved. Transplantation of thymic tissue (e.g., vascularized thymus or a thymokidney) can induce T cell tolerance by generating a T cell repertoire that is not reactive to a xenograft. The use of xenogeneic donors allows the possibility of using bone marrow cells and organs from the same animal, or from genetically matched animals. For bone marrow transplant, the recipient can be administered low dose radiation. In some cases, the recipient can be treated with an agent that depletes complement, such as cobra venom factor (e.g., at day −1).

### 8.2.1.4 Composite Islet-Kidney Graft

As provided here, kidneys from the genetically modified swine in which human CD47 is expressed in glomeruli of the kidney at higher levels than in renal tubules of the kidney can be used as a xenograft for xenotransplantion into human patients. In some embodiments, the xenograft can be include a combination of a kidney, such as a kidney described in Section 8.1, and islet of Langerhans cells. For example, the islet cells can be combined with the kidney of the present disclosure to generate a composite islet-kidney graft.

Generation of a composite islet-kidney graft can be performed by any method known in the art. By way of example, a partial pancreatectomy can be performed and the islet cells isolated. Thereafter, the islet cells can be combined with a kidney to form a composite islet-kidney cell that can then be used for xenotransplantation. See, e.g., Pomposelli et al., Front Endocrinol (Lausanne). May 12, 12:632605 (2021).

Accordingly, in a specific embodiment, the xenograft is a xenograft from a non-human species, wherein the xenograft comprises: (a) a kidney; and (b) islet of Langerhans cells, wherein the kidney comprises glomeruli that express human CD47 at a level higher than the level of human CD47 expression in the tubules of the kidney.

### 8.2.2. Effects of Treatments

In some embodiments, a method of transplantation described herein results in decreased risk or intensity of proteinuria, see section 8.2.4 below. In some embodiments, a method of transplantation described herein results in decreased occurrences of rejection of the donor kidney compared to methods of transplantation wherein the glomeruli of the donor kidney do not express human CD47 at a level higher than the level of human CD47 expression in the tubules of the donor kidney.

In some embodiments, the method results in reduced administration (e.g., administration reduced by about 10%, 10-20%, 20-30%, 30-40%, 40-50%, 50-60%, 60-70%, 70-80%, 80-90% or by over 90%) of immunosuppressive therapy to the recipient compared to a recipient of a donor kidney wherein the glomeruli of the donor kidney do not express human CD47 at a level higher than the level of human CD47 expression in the tubules of the donor kidney. In specific embodiments, the method results in reduced administration (e.g., administration reduced by about 10%, 10-20%, 20-30%, 30-40%, 40-50%, 50-60%, 60-70%, 70-80%, 80-90% or by over 90%) of immunosuppressive therapy to the recipient compared to the amount of immunosuppressive therapy which is typically administered to a comparable recipient (e.g., a person of the same sex and of comparable age, height, and/or weight), wherein the comparable recipient has received donor kidney wherein the glomeruli of the donor kidney do not express human CD47 at a level higher than the level of human CD47 expression in the tubules of the donor kidney. In other embodiments, the method results in reduced administration (e.g., administration reduced by about 10%, 10-20%, 20-30%, 30-40%, 40-50%, 50-60%, 60-70%, 70-80%, 80-90% or by over 90%) of immunosuppressive therapy to the recipient compared to the amount of immunosuppressive therapy which said recipient required after receipt of a prior donor kidney wherein the glomeruli of the donor kidney do not express human CD47 at a level higher than the level of human CD47 expression in the tubules of the donor kidney. In some embodiments, method results in the recipient requiring no further administration of immunosuppressive therapy, e.g., an immunosuppressive therapy described in section 8.2.1.1 below.

In some embodiments, the method results in about 10% reduction of immunosuppressive therapy. In some embodiments, the method results in about 10% to about 20% reduction of immunosuppressive therapy. In some embodiments, the method results in about 20% to about 30% reduction of immunosuppressive therapy. In some embodiments, the method results in about 30% to about 40% reduction of immunosuppressive therapy. In some embodiments, the method results in about 40% to about 50% reduction of immunosuppressive therapy. In some embodiments, the method results in about 50% to about 60% reduction of immunosuppressive therapy. In some embodiments, the method results in about 60% to about 70% reduction of immunosuppressive therapy. In some embodiments, the method results in about 70% to about 80% reduction of immunosuppressive therapy. In some embodiments, the method results in about 80% to about 90% reduction of immunosuppressive therapy. In some embodiments, the method results in more than about 90% reduction of immunosuppressive therapy.

In some embodiments, the method results in prolonged viability of the donor kidney, compared to a donor kidney wherein the glomeruli of the donor kidney do not express human CD47 at a level higher than the level of human CD47 expression in the tubules of the donor kidney. In some embodiments, the method results in prolonged viability (e.g., viability prolonged about 10%, 10-20%, 20-30%, 30-40%, 40-50%, 50-75%, 75-100%, 100-200%, 200-300% or by over 300%; or prolonged by 1-2 years, 2-3 years, 3-4 years, 4-5 years, 5-6 years, 6-8 years, 8-10 years, 10-15 years or 15-20 years) of the donor kidney compared to a donor kidney wherein the glomeruli of the donor kidney do not express human CD47 at a level higher than the level of human CD47 expression in the tubules of the donor kidney transplanted into a comparable recipient (e.g., a patient of the same sex and of comparable age, height, and/or weight). In some embodiments, the method results in prolonged viability (e.g., viability prolonged about 10%, 10-20%, 20-30%, 30-40%, 40-50%, 50-75%, 75-100%, 100-200%, 200-300% or by over 300%; or prolonged by 1-2 years, 2-3 years, 3-4 years, 4-5 years, 5-6 years, 6-8 years, 8-10 years, 10-15 years or 15-20 years) of the donor kidney compared to the viability of a donor kidney which said recipient has previously received, wherein the glomeruli of the donor kidney do not express human CD47 at a level higher than the level of human CD47 expression in the tubules of the donor kidney.

In some embodiments, viability of the donor kidney is prolonger about 10%, compared to a donor kidney wherein the glomeruli of the donor kidney do not express human CD47 at a level higher than the level of human CD47 expression in the tubules of the donor kidney. In some embodiments, viability is prolonged about 10-20%. In some embodiments, viability is prolonged about 20-30%. In some embodiments, viability is prolonged about 30-40%, In some embodiments, viability is prolonged about 40-50%. In some embodiments, viability is prolonged about 50-75%. In some embodiments, viability is prolonged about 75-100%. In some embodiments, viability is prolonged about 100-200%. In some embodiments, viability is prolonged about 200-300%. In some embodiments, viability is prolonged over 300%.

In some embodiments, viability of the donor kidney is prolonged by 1-2 years, compared to a donor kidney wherein the glomeruli of the donor kidney do not express human CD47 at a level higher than the level of human CD47 expression in the tubules of the donor kidney. In some embodiments, viability of the donor kidney is prolonged 2-3 years. In some embodiments, viability of the donor kidney is prolonged 3-4 years. In some embodiments, viability of the donor kidney is prolonged 4-5 years. In some embodiments, viability of the donor kidney is prolonged 5-6 years. In some embodiments, viability of the donor kidney is prolonged 6-8 years. In some embodiments, viability of the donor kidney is prolonged 8-10 years. In some embodiments, viability of the donor kidney is prolonged 10-15 years. In some embodiments, viability of the donor kidney is prolonged 15-20 years.

In some embodiments, the method results in better quality of life for the recipient compared to a recipient of a donor kidney wherein the glomeruli of the donor kidney do not express human CD47 at a level higher than the level of human CD47 expression in the tubules of the donor kidney. In other embodiments, the method results in better quality of life for the recipient compared to a comparable recipient (e.g., a person of the same sex and of comparable age, height, and/or weight), wherein the comparable recipient has received a donor kidney wherein the glomeruli of the donor kidney do not express human CD47 at a level higher than the level of human CD47 expression in the tubules of the donor kidney. In other embodiments, the method results in better quality of life for the recipient compared to the quality of life said recipient experienced after a prior transplantation with a donor kidney wherein the glomeruli of the donor kidney did not express human CD47 at a level higher than the level of human CD47 expression in the tubules of the donor kidney.

In some embodiments, the method results in longer survival (e.g., 10-20%, 20-30%, 40-50%, 50-60%, 60-70%, 70-80%, 80-90%, or 90-100% longer; or 2 to 3-fold, 3 to 5-fold, 5 to 7-fold, 7 to 10-fold or 10 to 15-fold longer) of the transplant recipient compared to a recipient of a donor kidney wherein the glomeruli of the donor kidney do not express human CD47 at a level higher than the level of human CD47 expression in the tubules of the donor kidney. In other embodiments, the method results in longer survival (e.g., 10-20%, 20-30%, 40-50%, 50-60%, 60-70%, 70-80%, 80-90%, or 90-100% longer; or 2 to 3-fold, 3 to 5-fold, 5 to 7-fold, 7 to 10-fold or 10 to 15-fold longer) of the transplant recipient compared to the survival of a comparable recipient (e.g., a person of the same sex and of comparable age, height, and/or weight), wherein the comparable recipient has received a donor kidney wherein the glomeruli of the donor kidney do not express human CD47 at a level higher than the level of human CD47 expression in the tubules of the donor kidney.

In some embodiments, the method results in 10-20% longer survival of the transplant recipient compared to a recipient of a donor kidney wherein the glomeruli of the donor kidney do not express human CD47 at a level higher than the level of human CD47 expression in the tubules of the donor kidney. In some embodiments, the method results in 20-30% longer survival of the transplant recipient. In some embodiments, the method results in 30-40% longer survival of the transplant recipient. In some embodiments, the method results in 50-60% longer survival of the transplant recipient. In some embodiments, the method results in 60-70% longer survival of the transplant recipient. In some embodiments, the method results in 70-80% longer survival of the transplant recipient. In some embodiments, the method results in 80-90% longer survival of the transplant recipient. In some embodiments, the method results in 90-100% longer survival of the transplant recipient.

In some embodiments, the method results in 2 to 3 fold longer survival of the transplant recipient compared to a recipient of a donor kidney wherein the glomeruli of the donor kidney do not express human CD47 at a level higher than the level of human CD47 expression in the tubules of the donor kidney. In some embodiments, the method results in 3 to 5-fold longer survival of the transplant recipient. In some embodiments, the method results in 5 to 7-fold longer survival of the transplant recipient. In some embodiments, the method results in 7 to 10-fold longer survival of the transplant recipient. In some embodiments, the method results in 10 to 15-fold longer survival of the transplant recipient.

### 8.2.3. Patient Population

In a preferred embodiment, a patient treated in accordance with the methods described herein (e.g., the recipient of one or more donor grafts) is a human patient. As used herein, the terms “subject” and “patient” are used interchangeably and include any human or non-human mammal. Non-limiting examples include members of the human, equine, porcine, bovine, rattus, murine, canine and feline species. In some embodiments, the subject is a non-human primate. In some embodiments, the subject is human. In specific embodiments, the subject is a human adult. In some embodiments, the subject is a human child. In specific embodiments, the subject is human and receives one or more donor grafts from a porcine donor. In other specific embodiments, the subject is a non-human primate (e.g., a baboon, a cynomolgus monkey or a rhesus macaque) and receives one or more grafts from a porcine donor.

In one aspect, a patient treated in accordance with the methods described herein is in need of a kidney transplant. A patient may be in need of a kidney transplant due to renal failure or the rejection of a donor kidney. Renal failure can have a number of causes, including but not limited to high blood pressure (hypertension), physical injury, diabetes, kidney disease (polycystic kidney disease, glomerular disease) and autoimmune disorders such as lupus. Renal failure may be acute or chronic. Kidney failure can also be diagnosed by laboratory tests such as glomerular filtration rate, blood urea nitrogen, and serum creatinine, by imaging test (ultrasound, computer tomography) or a kidney biopsy.

In some embodiments, a patient treated in accordance with a method described herein has Stage 1 kidney disease. In some embodiments, a patient treated in accordance with a method described herein has Stage 2 kidney disease. In some embodiments, a patient treated in accordance with a method described herein has Stage 3 kidney disease. In some embodiments, a patient treated in accordance with a method described herein has Stage 4 kidney disease. In some embodiments, a patient treated in accordance with a method described herein has Stage 5 kidney disease.

In some embodiments, a patient treated in accordance with a method described herein has a glomerular filtration rate (GFR) of about 90 or higher. In some embodiments, a patient treated in accordance with a method described herein has a GFR of about 60-90. In some embodiments, a patient treated in accordance with a method described herein has a GFR of about 30-60. In some embodiments, a patient treated in accordance with a method described herein has a GFR of about 15-30. In some embodiments, a patient treated in accordance with a method described herein has a GFR of about 15 or less.

### 8.2.4. Methods For Preventing or Reducing the Severity of Proteinuria

Proteinuria is characterized by increased levels of protein in the urine and can be a symptom of decreased kidney function and potentially renal failure. It is commonly caused by glomerular disease which results in loss of albumin and immunoglobulins in the urine. Proteinuria can also be caused by tubular disease and other renal diseases, as well as certain drugs. See e.g., Carroll and Temte, Am Fam Physician 62(6):1333-1340 (2000) and BMJ Best Practice: Evaluation of Proteinuria [online] [retrieved on Aug. 26, 2020], retrieved from the internet:<URL: https://bestpractice.bmj.com/topics/en-us/875>. In addition, proteinuria often occurs after a kidney transplantation. Proteinuria of 500 mg per day or less (e.g., 200-500 mg per day) at one year post transplantation correlates with poor outcome (e.g., graft rejection). See, e.g., Diena et al. (2019), BMC Nephrology 20:443 and Kang et al. (2009) J Korean Med Sci. 24 (Suppl 1): S129-34.

Protein excretion of more than 150 mg per day is a commonly a used as a diagnosis for proteinuria. Dipstick analysis is often used to measure protein concentrations in the urine. This is a semi-quantitative method, the results of which are expressed as negative, trace, 1+, 2+, 3+ or 4+. See e.g., Carroll and Temte, Am Fam Physician 62(6):1333-1340 (2000). Total protein levels or only albumin levels may be measured to provide a quantitative test. Results may be expressed in total protein or albumin levels, or in alumni to creatine ration or protein to creatine ratio. Proteinuria that persists for over three months is a diagnostic criteria of chronic kidney disease. Conversely, reduction of proteinuria is used as a surrogate marker in the management of chronic kidney diseases. See, e.g., BMJ Best Practice: Evaluation of Proteinuria [online] [retrieved on Aug. 26, 2020], retrieved from the internet<URL: https://bestpractice.bmj.com/topics/en-us/875>.

In one aspect, the methods of transplantation described herein (such as the methods of transplanting bone marrow from a first donor swine a kidney from a second donor swine, or methods of transplanting bone marrow and a kidney from one donor swine as described in section 8.2 above), result in reduced risk, severity or duration of proteinuria. In particular embodiments, the methods of transplantation described herein (e.g., the methods described in section 8.2 above) wherein the glomeruli of the donor kidney express human CD47 at a level higher than the level of human CD47 expression in the tubules of the donor kidney result in a reduced severity of proteinuria. In particular embodiments, the methods of transplantation described herein (e.g., the methods described in section 8.2 above) wherein the glomeruli of the donor kidney express human CD47 at a level higher than the level of human CD47 expression in the tubules of the donor kidney result in a reduced duration of proteinuria. In particular embodiments, the methods of transplantation described herein (e.g., the methods described in section 8.2 above) wherein the glomeruli of the donor kidney express human CD47 at a level higher than the level of human CD47 expression in the tubules of the donor kidney result in a reduced risk of proteinuria in a treated population. For example, the severity of proteinuria in a patient treated in accordance with the methods herein may be decreased compared to the severity of proteinuria observed in a patient receiving a donor kidney wherein the glomeruli of the donor kidney do not express human CD47 at a level higher than the level of human CD47 expression in the tubules of the donor kidney.

In some embodiments, the severity of proteinuria, as measured by protein levels in the urine, is reduced by 10%, 20%, 30%, 40%, 50%, 60%, 70%, 80%, 90%, 95% or over 95%. In some embodiments, the severity of proteinuria, as measured by protein levels in the urine, is reduced by 10%. In some embodiments, the severity of proteinuria, as measured by protein levels in the urine, is reduced by 20%. In some embodiments, the severity of proteinuria, as measured by protein levels in the urine, is reduced by 30%. In some embodiments, the severity of proteinuria, as measured by protein levels in the urine, is reduced by 40%. In some embodiments, the severity of proteinuria, as measured by protein levels in the urine, is reduced by 50%. In some embodiments, the severity of proteinuria, as measured by protein levels in the urine, is reduced by 60%. In some embodiments, the severity of proteinuria, as measured by protein levels in the urine, is reduced by 70%. In some embodiments, the severity of proteinuria, as measured by protein levels in the urine, is reduced by 80%. In some embodiments, the severity of proteinuria, as measured by protein levels in the urine, is reduced by 90%. In some embodiments, the severity of proteinuria, as measured by protein levels in the urine, is reduced by over 95%.

In some embodiments, a patient treated in accordance with a method provided herein will not experience proteinuria, defined as the excretion or over 150 mg protein per day in the urine. In some embodiments, a patient treated in accordance with a method provided herein may experience transient proteinuria that resolves after 1, 2, 3, 3-7, 7-10, 10-14 days, or 1-2, 2-3, 3-4, 4-5, 5-6, 6-7, 7-8 weeks, or 1, 2, 3, 4, 5, 6 months after the transplantation.

In some embodiments, the concentration of total protein in the urine of a recipient treated with a method described herein developing proteinuria is less than about 60 mg per day, less than about 80 mg per day, less than about 100 mg per day, less than about 120 mg per day, less than about 140 mg per day, less than about 160 mg per day, less than about 200 mg per day, less than about 220 mg per day, less than about 240 mg, per day, less than about 260 mg per day, less than about 280 mg per day, less than about 300 mg per day, less than about 320 mg per day, less than about 340 mg per day, less than about 360 mg per day, less than about 380 mg per day or less than about 400 mg per day. In some embodiments, the concentration of total protein in the urine of a recipient treated with a method described herein developing proteinuria is less than about 60 mg per day. In some embodiments, the concentration of total protein in the urine of a recipient treated with a method described herein developing proteinuria is less than about 80 mg per day. In some embodiments, the concentration of total protein in the urine of a recipient treated with a method described herein developing proteinuria is less than about 100 mg per day. In some embodiments, the concentration of total protein in the urine of a recipient treated with a method described herein developing proteinuria is less than about 120 mg per day. In some embodiments, the concentration of total protein in the urine of a recipient treated with a method described herein developing proteinuria is less than about 140 mg per day. In some embodiments, the concentration of total protein in the urine of a recipient treated with a method described herein developing proteinuria is less than about 160 mg per day. In some embodiments, the concentration of total protein in the urine of a recipient treated with a method described herein developing proteinuria is less than about 200 mg per day. In some embodiments, the concentration of total protein in the urine of a recipient treated with a method described herein developing proteinuria is less than about 220 mg per day. In some embodiments, the concentration of total protein in the urine of a recipient treated with a method described herein developing proteinuria is less than about 240 mg per day. In some embodiments, the concentration of total protein in the urine of a recipient treated with a method described herein developing proteinuria is less than about 260 mg per day. In some embodiments, the concentration of total protein in the urine of a recipient treated with a method described herein developing proteinuria is less than about 280 mg per day. In some embodiments, the concentration of total protein in the urine of a recipient treated with a method described herein developing proteinuria is less than about 300 mg per day. In some embodiments, the concentration of total protein in the urine of a recipient treated with a method described herein developing proteinuria is less than about 320 mg per day. In some embodiments, the concentration of total protein in the urine of a recipient treated with a method described herein developing proteinuria is less than about 340 mg per day. In some embodiments, the concentration of total protein in the urine of a recipient treated with a method described herein developing proteinuria is less than about 360 mg per day. In some embodiments, the concentration of total protein in the urine of a recipient treated with a method described herein developing proteinuria is less than about 380 mg per day. In some embodiments, the concentration of total protein in the urine of a recipient treated with a method described herein developing proteinuria is less than about 400 mg per day.

In some embodiments, the concentration of albumin in the urine of a recipient treated with a method described herein developing proteinuria is less than about 5 mg per day, less than about 10 mg per day, less than about 20 mg per day, less than about 30 mg per day, less than about 40 mg per day, less than about 50 mg per day, less than about 60 mg per day, less than about 70 mg per day, less than about 80 mg per day, less than about 90 mg per day or less than about 100 mg per day. In some embodiments, the concentration of albumin in the urine of a recipient treated with a method described herein developing proteinuria is less than about 5 mg per day. In some embodiments, the concentration of albumin in the urine of a recipient treated with a method described herein developing proteinuria is less than about 10 mg per day. In some embodiments, the concentration of albumin in the urine of a recipient treated with a method described herein developing proteinuria is less than about 20 mg per day. In some embodiments, the concentration of albumin in the urine of a recipient treated with a method described herein developing proteinuria is less than about 30 mg per day. In some embodiments, the concentration of albumin in the urine of a recipient treated with a method described herein developing proteinuria is less than about 40 mg per day. In some embodiments, the concentration of albumin in the urine of a recipient treated with a method described herein developing proteinuria is less than about 50 mg per day. In some embodiments, the concentration of albumin in the urine of a recipient treated with a method described herein developing proteinuria is less than about 60 mg per day. In some embodiments, the concentration of albumin in the urine of a recipient treated with a method described herein developing proteinuria is less than about 70 mg per day. In some embodiments, the concentration of albumin in the urine of a recipient treated with a method described herein developing proteinuria is less than about 80 mg per day. In some embodiments, the concentration of albumin in the urine of a recipient treated with a method described herein developing proteinuria is less than about 90 mg per day. In some embodiments, the concentration of albumin in the urine of a recipient treated with a method described herein developing proteinuria is less than about 100 mg per day.

In some embodiments, the ratio of protein to creatinine in a 24 hour urine sample of a patient treated in accordance with the methods described herein is less than about 0.2, less than about 0.4, less than about 0.6, less than about 0.8 or less than about 1. In some embodiments, the ratio of albumin to creatinine in a 24 hour urine sample of a patient treated in accordance with the methods described herein is less than about 0.02, less than about 0.04, less than about 0.06, less than about 0.08 or less than about 0.1. In some embodiments, the ratio of protein to creatinine in a 24 hour urine sample of a patient treated in accordance with the methods described herein is less than about 0.2. In some embodiments, the ratio of protein to creatinine in a 24 hour urine sample of a patient treated in accordance with the methods described herein is less than about 0.4. In some embodiments, the ratio of protein to creatinine in a 24 hour urine sample of a patient treated in accordance with the methods described herein is less than about 0.6. In some embodiments, the ratio of protein to creatinine in a 24 hour urine sample of a patient treated in accordance with the methods described herein is less than about 0.8. In some embodiments, the ratio of protein to creatinine in a 24 hour urine sample of a patient treated in accordance with the methods described herein is less than about 1.0. In some embodiments, the ratio of albumin to creatinine in a 24 hour urine sample of a patient treated in accordance with the methods described herein is less than about 0.02. In some embodiments, the ratio of albumin to creatinine in a 24 hour urine sample of a patient treated in accordance with the methods described herein is less than about 0.04. In some embodiments, the ratio of albumin to creatinine in a 24 hour urine sample of a patient treated in accordance with the methods described herein is less than about 0.06. In some embodiments, the ratio of albumin to creatinine in a 24 hour urine sample of a patient treated in accordance with the methods described herein is less than about 0.08. In some embodiments, the ratio of albumin to creatinine in a 24 hour urine sample of a patient treated in accordance with the methods described herein is less than about 0.1.

In some embodiments, the risk of a recipient treated with a method described herein developing proteinuria is decreased by about 10%, 20%, 30%, 40%, 50%, 60%, 70%, 80%, 90% or 95% compared to the risk of a recipient of a donor kidney wherein the glomeruli of the donor kidney do not express human CD47 at a level higher than the level of human CD47 expression in the tubules of the donor kidney. In some embodiments, the risk is decreased by about 10%. In some embodiments, the risk is decreased by about 20%. In some embodiments, the risk is decreased by about 30%. In some embodiments, the risk is decreased by about 40%. In some embodiments, the risk is decreased by about 50%. In some embodiments, the risk is decreased by about 60%. In some embodiments, the risk is decreased by about 70%. In some embodiments, the risk is decreased by about 80%. In some embodiments, the risk is decreased by about 90%. In some embodiments, the risk is decreased by about 95%.

TABLE 1
Table of Sequences

| Name | SEQ ID NO: | Sequence |
|---|---|---|
| leukocyte surface | 1 | MWPLVAALLLGSACCGSAQLLFNKTKSVEFTFCNDTVVIPCFVT |
| antigen CD47 | | NMEAQNTTEVYVKWKFKGRDIYTFDGALNKSTVPTDFSSAKIEV |
| isoform 1 precursor | | SQLLKGDASLKMDKSDAVSHTGNYTCEVTELTREGETIIELKYRV |
| [Homo sapiens] | | VSWFSPNENILIVIFPIFAILLFWGQFGIKTLKYRSGGMDEKTIALL |
| (NCBI Reference | | VAGLVITVIVIVGAILFVPGEYSLKNATGLGLIVTSTGILILLHYYV |
| Sequence: | | FSTAIGLTSFVIAILVIQVIAYILAVVGLSLCIAACIPMHGPLLISGLS |
| NP_001768.1 | | ILALAQLLGLVYMKFVASNQKTIQPPRKAVEEPLNAFKESKGMM |
| | | NDE |
| CD47 molecule | 2 | MWPLVAALLLGSACCGSAQLLFNKTKSVEFTFCNDTVVIPCFVT |
| [Homo sapiens | | NMEAQNTTEVYVKWKFKGRDIYTFDGALNKSTVPTDFSSAKIEV |
| (human)] | | SQLLKGDASLKMDKSDAVSHTGNYTCEVTELTREGETIIELKYRV |
| NP_942088 | | VSWFSPNENILIVIFPIFAILLFWGQFGIKTLKYRSGGMDEKTIALL |
| | | VAGLVITVIVIVGAILFVPGEYSLKNATGLGLIVTSTGILILLHYYV |
| | | FSTAIGLTSFVIAILVIQVIAYILAVVGLSLCIAACIPMHGPLLISGLS |
| | | ILALAQLLGLVYMKFVASNQKTIQPPRNN |
| Homo sapiens CD47 | 3 | GCAGCCTGGGCAGTGGGTCCTGCCTGTGACGCGCGGCGGCGG |
| molecule (CD47), | | TCGGTCCTGCCTGTAACGGCGGCGGCGGCTGCTGCTCCGGACA |
| transcript variant 1, | | CCTGCGGCGGCGGCGGCGACCCCGCGGCGGGCGCGGAGATGT |
| mRNA | | GGCCCCTGGTAGCGGCGCTGTTGCTGGGCTCGGCGTGCTGCGG |
| NM_001777 | | ATCAGCTCAGCTACTATTTAATAAAACAAAATCTGTAGAATTC |
| | | ACGTTTTGTAATGACACTGTCGTCATTCCATGCTTTGTTACTAA |
| | | TATGGAGGCACAAAACACTACTGAAGTATACGTAAAGTGGAA |
| | | ATTTAAAGGAAGAGATATTTACACCTTTGATGGAGCTCTAAAC |
| | | AAGTCCACTGTCCCCACTGACTTTAGTAGTGCAAAAATTGAAG |
| | | TCTCACAATTACTAAAAGGAGATGCCTCTTTGAAGATGGATAA |
| | | GAGTGATGCTGTCTCACACACAGGAAACTACACTTGTGAAGTA |
| | | ACAGAATTAACCAGAGAAGGTGAAACGATCATCGAGCTAAAA |
| | | TATCGTGTTGTTTCATGGTTTTCTCCAAATGAAAATATTCTTAT |
| | | TGTTATTTTCCCAATTTTTGCTATACTCCTGTTCTGGGGACAGT |
| | | TTGGTATTAAAACACTTAAATATAGATCCGGTGGTATGGATGA |
| | | GAAAACAATTGCTTTACTTGTTGCTGGACTAGTGATCACTGTC |
| | | ATTGTCATTGTTGGAGCCATTCTTTTCGTCCCAGGTGAATATTC |
| | | ATTAAAGAATGCTACTGGCCTTGGTTTAATTGTGACTTCTACA |
| | | GGGATATTAATATTACTTCACTACTATGTGTTTAGTACAGCGA |
| | | TTGGATTAACCTCCTTCGTCATTGCCATATTGGTTATTCAGGTG |
| | | ATAGCCTATATCCTCGCTGTGGTTGGACTGAGTCTCTGTATTGC |
| | | GGCGTGTATACCAATGCATGGCCCTCTTCTGATTTCAGGTTTG |
| | | AGTATCTTAGCTCTAGCACAATTACTTGGACTAGTTTATATGA |
| | | AATTTGTGGCTTCCAATCAGAAGACTATACAACCTCCTAGGAA |
| | | AGCTGTAGAGGAACCCCTTAATGCATTCAAAGAATCAAAAGG |
| | | AATGATGAATGATGAATAACTGAAGTGAAGTGATGGACTCCG |
| | | ATTTGGAGAGTAGTAAGACGTGAAAGGAATACACTTGTGTTTA |
| | | AGCACCATGGCCTTGATGATTCACTGTTGGGGAGAAGAAACA |
| | | AGAAAAGTAACTGGTTGTCACCTATGAGACCCTTACGTGATTG |
| | | TTAGTTAAGTTTTTATTCAAAGCAGCTGTAATTTAGTTAATAAA |
| | | ATAATTATGATCTATGTTGTTTGCCCAATTGAGATCCAGTTTTT |
| | | TGTTGTTATTTTTAATCAATTAGGGGCAATAGTAGAATGGACA |
| | | ATTTCCAAGAATGATGCCTTTCAGGTCCTAGGGCCTCTGGCCT |
| | | CTAGGTAACCAGTTTAAATTGGTTCAGGGTGATAACTACTTAG |
| | | CACTGCCCTGGTGATTACCCAGAGATATCTATGAAAACCAGTG |
| | | GCTTCCATCAAACCTTTGCCAACTCAGGTTCACAGCAGCTTTG |
| | | GGCAGTTATGGCAGTATGGCATTAGCTGAGAGGTGTCTGCCAC |
| | | TTCTGGGTCAATGGAATAATAAATTAAGTACAGGCAGGAATTT |
| | | GGTTGGGAGCATCTTGTATGATCTCCGTATGATGTGATATTGA |
| | | TGGAGATAGTGGTCCTCATTCTTGGGGGTTGCCATTCCCACAT |
| | | TCCCCCTTCAACAAACAGTGTAACAGGTCCTTCCCAGATTTAG |
| | | GGTACTTTTATTGATGGATATGTTTTCCTTTTATTCACATAACC |
| | | CCTTGAAACCCTGTCTTGTCCTCCTGTTACTTGCTTCTGCTGTA |
| | | CAAGATGTAGCACCTTTTCTCCTCTTTGAACATGGTCTAGTGA |
| | | CACGGTAGCACCAGTTGCAGGAAGGAGCCAGACTTGTTCTCA |
| | | GAGCACTGTGTTCACACTTTTCAGCAAAAATAGCTATGGTTGT |
| | | AACATATGTATTCCCTTCCTCTGATTTGAAGGCAAAAATCTAC |
| | | AGTGTTTCTTCACTTCTTTTCTGATCTGGGGCATGAAAAAAGC |
| | | AAGATTGAAATTTGAACTATGAGTCTCCTGCATGGCAACAAAA |
| | | TGTGTGTCACCATCAGGCCAACAGGCCAGCCCTTGAATGGGG |
| | | ATTTATTACTGTTGTATCTATGTTGCATGATAAACATTCATCAC |
| | | CTTCCTCCTGTAGTCCTGCCTCGTACTCCCCTTCCCCTATGATT |
| | | GAAAAGTAAACAAAACCCACATTTCCTATCCTGGTTAGAAGA |
| | | AAATTAATGTTCTGACAGTTGTGATCGCCTGGAGTACTTTTAG |
| | | ACTTTTAGCATTCGTTTTTTACCTGTTTGTGGATGTGTGTTTGT |
| | | ATGTGCATACGTATGAGATAGGCACATGCATCTTCTGTATGGA |
| | | CAAAGGTGGGGTACCTACAGGAGAGCAAAGGTTAATTTTGTG |
| | | CTTTTAGTAAAAACATTTAAATACAAAGTTCTTTATTGGGTGG |
| | | AATTATATTTGATGCAAATATTTGATCACTTAAAACTTTTAAA |
| | | ACTTCTAGGTAATTTGCCACGCTTTTTGACTGCTCACCAATACC |
| | | CTGTAAAAATACGTAATTCTTCCTGTTTGTGTAATAAGATATTC |
| | | ATATTTGTAGTTGCATTAATAATAGTTATTTCTTAGTCCATCAG |
| | | ATGTTCCCGTGTGCCTCTTTTATGCCAAATTGATTGTCATATTT |
| | | CATGTTGGGACCAAGTAGTTTGCCCATGGCAAACCTAAATTTA |
| | | TGACCTGCTGAGGCCTCTCAGAAAACTGAGCATACTAGCAAG |
| | | ACAGCTCTTCTTGAAAAAAAAAATATGTATACACAAATATATA |
| | | CGTATATCTATATATACGTATGTATATACACACATGTATATTCT |
| | | TCCTTGATTGTGTAGCTGTCCAAAATAATAACATATATAGAGG |
| | | GAGCTGTATTCCTTTATACAAATCTGATGGCTCCTGCAGCACT |
| | | TTTTCCTTCTGAAAATATTTACATTTTGCTAACCTAGTTTGTTA |
| | | CTTTAAAAATCAGTTTTGATGAAAGGAGGGAAAAGCAGATGG |
| | | ACTTGAAAAAGATCCAAGCTCCTATTAGAAAAGGTATGAAAA |
| | | TCTTTATAGTAAAATTTTTTATAAACTAAAGTTGTACCTTTTAA |
| | | TATGTAGTAAACTCTCATTTATTTGGGGTTCGCTCTTGGATCTC |
| | | ATCCATCCATTGTGTTCTCTTTAATGCTGCCTGCCTTTTGAGGC |
| | | ATTCACTGCCCTAGACAATGCCACCAGAGATAGTGGGGGAAA |
| | | TGCCAGATGAAACCAACTCTTGCTCTCACTAGTTGTCAGCTTC |
| | | TCTGGATAAGTGACCACAGAAGCAGGAGTCCTCCTGCTTGGGC |
| | | ATCATTGGGCCAGTTCCTTCTCTTTAAATCAGATTTGTAATGGC |
| | | TCCCAAATTCCATCACATCACATTTAAATTGCAGACAGTGTTT |
| | | TGCACATCATGTATCTGTTTTGTCCCATAATATGCTTTTTACTC |
| | | CCTGATCCCAGTTTCTGCTGTTGACTCTTCCATTCAGTTTTATT |
| | | TATTGTGTGTTCTCACAGTGACACCATTTGTCCTTTTCTGCAAC |
| | | AACCTTTCCAGCTACTTTTGCCAAATTCTATTTGTCTTCTCCTT |
| | | CAAAACATTCTCCTTTGCAGTTCCTCTTCATCTGTGTAGCTGCT |
| | | CTTTTGTCTCTTAACTTACCATTCCTATAGTACTTTATGCATCT |
| | | CTGCTTAGTTCTATTAGTTTTTTGGCCTTGCTCTTCTCCTTGATT |
| | | TTAAAATTCCTTCTATAGCTAGAGCTTTTCTTTCTTTCATTCTCT |
| | | CTTCCTGCAGTGTTTTGCATACATCAGAAGCTAGGTACATAAG |
| | | TTAAATGATTGAGAGTTGGCTGTATTTAGATTTATCACTTTTTA |
| | | ATAGGGTGAGCTTGAGAGTTTTCTTTCTTTCTGTTTTTTTTTTTT |
| | | GTTTTTTTTTTTTTTTTTTTTTTTTTTTTTTTTGACTAATTTCACA |
| | | TGCTCTAAAAACCTTCAAAGGTGATTATTTTTCTCCTGGAAAC |
| | | TCCAGGTCCATTCTGTTTAAATCCCTAAGAATGTCAGAATTAA |
| | | AATAACAGGGCTATCCCGTAATTGGAAATATTTCTTTTTTCAG |
| | | GATGCTATAGTCAATTTAGTAAGTGACCACCAAATTGTTATTT |
| | | GCACTAACAAAGCTCAAAACACGATAAGTTTACTCCTCCATCT |
| | | CAGTAATAAAAATTAAGCTGTAATCAACCTTCTAGGTTTCTCT |
| | | TGTCTTAAAATGGGTATTCAAAAATGGGGATCTGTGGTGTATG |
| | | TATGGAAACACATACTCCTTAATTTACCTGTTGTTGGAAACTG |
| | | GAGAAATGATTGTCGGGCAACCGTTTATTTTTTATTGTATTTTA |
| | | TTTGGTTGAGGGATTTTTTTATAAACAGTTTTACTTGTGTCATA |
| | | TTTTAAAATTACTAACTGCCATCACCTGCTGGGGTCCTTTGTTA |
| | | GGTCATTTTCAGTGACTAATAGGGATAATCCAGGTAACTTTGA |
| | | AGAGATGAGCAGTGAGTGACCAGGCAGTTTTTCTGCCTTTAGC |
| | | TTTGACAGTTCTTAATTAAGATCATTGAAGACCAGCTTTCTCAT |
| | | AAATTTCTCTTTTTGAAAAAAAGAAAGCATTTGTACTAAGCTC |
| | | CTCTGTAAGACAACATCTTAAATCTTAAAAGTGTTGTTATCAT |
| | | GACTGGTGAGAGAAGAAAACATTTTGTTTTTATTAAATGGAGC |
| | | ATTATTTACAAAAAGCCATTGTTGAGAATTAGATCCCACATCG |
| | | TATAAATATCTATTAACCATTCTAAATAAAGAGAACTCCAGTG |
| | | TTGCTATGTGCAAGATCCTCTCTTGGAGCTTTTTTGCATAGCAA |
| | | TTAAAGGTGTGCTATTTGTCAGTAGCCATTTTTTTGCAGTGATT |
| | | TGAAGACCAAAGTTGTTTTACAGCTGTGTTACCGTTAAAGGTT |
| | | TTTTTTTTTATATGTATTAAATCAATTTATCACTGTTTAAAGCTT |
| | | TGAATATCTGCAATCTTTGCCAAGGTACTTTTTTATTTAAAAAA |
| | | AAACATAACTTTGTAAATATTACCCTGTAATATTATATATACTT |
| | | AATAAAACATTTTAAGCTATTTTGTTGGGCTATTTCTATTGCTG |
| | | CTACAGCAGACCACAAGCACATTTCTGAAAAATTTAATTTATT |
| | | AATGTATTTTTAAGTTGCTTATATTCTAGGTAACAATGTAAAG |
| | | AATGATTTAAAATATTAATTATGAATTTTTTGAGTATAATACCC |
| | | AATAAGCTTTTAATTAGAGCAGAGTTTTAATTAAAAGTTTTAA |
| | | ATCAGTCCAA |
| Homo sapiens CD47 | 4 | GGGGAGCAGGCGGGGGAGCGGGCGGGAAGCAGTGGGAGCGC |
| molecule (CD47), | | GCGTGCGCGCGGCCGTGCAGCCTGGGCAGTGGGTCCTGCCTGT |
| transcript variant 2, | | GACGCGCGGCGGCGGTCGGTCCTGCCTGTAACGGCGGCGGCG |
| mRNA | | GCTGCTGCTCCAGACACCTGCGGCGGCGGCGGCGACCCCGCG |
| NCBI Reference | | GCGGGCGCGGAGATGTGGCCCCTGGTAGCGGCGCTGTTGCTG |
| Sequence: | | GGCTCGGCGTGCTGCGGATCAGCTCAGCTACTATTTAATAAAA |
| NM_198793.3 | | CAAAATCTGTAGAATTCACGTTTTGTAATGACACTGTCGTCAT |
| | | TCCATGCTTTGTTACTAATATGGAGGCACAAAACACTACTGAA |
| | | GTATACGTAAAGTGGAAATTTAAAGGAAGAGATATTTACACC |
| | | TTTGATGGAGCTCTAAACAAGTCCACTGTCCCCACTGACTTTA |
| | | GTAGTGCAAAAATTGAAGTCTCACAATTACTAAAAGGAGATG |
| | | CCTCTTTGAAGATGGATAAGAGTGATGCTGTCTCACACACAGG |
| | | AAACTACACTTGTGAAGTAACAGAATTAACCAGAGAAGGTGA |
| | | AACGATCATCGAGCTAAAATATCGTGTTGTTTCATGGTTTTCTC |
| | | CAAATGAAAATATTCTTATTGTTATTTTCCCAATTTTTGCTATA |
| | | CTCCTGTTCTGGGGACAGTTTGGTATTAAAACACTTAAATATA |
| | | GATCCGGTGGTATGGATGAGAAAACAATTGCTTTACTTGTTGC |
| | | TGGACTAGTGATCACTGTCATTGTCATTGTTGGAGCCATTCTTT |
| | | TCGTCCCAGGTGAATATTCATTAAAGAATGCTACTGGCCTTGG |
| | | TTTAATTGTGACTTCTACAGGGATATTAATATTACTTCACTACT |
| | | ATGTGTTTAGTACAGCGATTGGATTAACCTCCTTCGTCATTGCC |
| | | ATATTGGTTATTCAGGTGATAGCCTATATCCTCGCTGTGGTTG |
| | | GACTGAGTCTCTGTATTGCGGCGTGTATACCAATGCATGGCCC |
| | | TCTTCTGATTTCAGGTTTGAGTATCTTAGCTCTAGCACAATTAC |
| | | TTGGACTAGTTTATATGAAATTTGTGGCTTCCAATCAGAAGAC |
| | | TATACAACCTCCTAGGAATAACTGAAGTGAAGTGATGGACTCC |
| | | GATTTGGAGAGTAGTAAGACGTGAAAGGAATACACTTGTGTTT |
| | | AAGCACCATGGCCTTGATGATTCACTGTTGGGGAGAAGAAAC |
| | | AAGAAAAGTAACTGGTTGTCACCTATGAGACCCTTACGTGATT |
| | | GTTAGTTAAGTTTTTATTCAAAGCAGCTGTAATTTAGTTAATAA |
| | | AATAATTATGATCTATGTTGTTTGCCCAATTGAGATCCAGTTTT |
| | | TTGTTGTTATTTTTAATCAATTAGGGGCAATAGTAGAATGGAC |
| | | AATTTCCAAGAATGATGCCTTTCAGGTCCTAGGGCCTCTGGCC |
| | | TCTAGGTAACCAGTTTAAATTGGTTCAGGGTGATAACTACTTA |
| | | GCACTGCCCTGGTGATTACCCAGAGATATCTATGAAAACCAGT |
| | | GGCTTCCATCAAACCTTTGCCAACTCAGGTTCACAGCAGCTTT |
| | | GGGCAGTTATGGCAGTATGGCATTAGCTGAGAGGTGTCTGCCA |
| | | CTTCTGGGTCAATGGAATAATAAATTAAGTACAGGCAGGAATT |
| | | TGGTTGGGAGCATCTTGTATGATCTCCGTATGATGTGATATTG |
| | | ATGGAGATAGTGGTCCTCATTCTTGGGGGTTGCCATTCCCACA |
| | | TTCCCCCTTCAACAAACAGTGTAACAGGTCCTTCCCAGATTTA |
| | | GGGTACTTTTATTGATGGATATGTTTTCCTTTTATTCACATAAC |
| | | CCCTTGAAACCCTGTCTTGTCCTCCTGTTACTTGCTTCTGCTGT |
| | | ACAAGATGTAGCACCTTTTCTCCTCTTTGAACATGGTCTAGTG |
| | | ACACGGTAGCACCAGTTGCAGGAAGGAGCCAGACTTGTTCTC |
| | | AGAGCACTGTGTTCACACTTTTCAGCAAAAATAGCTATGGTTG |
| | | TAACATATGTATTCCCTTCCTCTGATTTGAAGGCAAAAATCTA |
| | | CAGTGTTTCTTCACTTCTTTTCTGATCTGGGGCATGAAAAAAG |
| | | CAAGATTGAAATTTGAACTATGAGTCTCCTGCATGGCAACAAA |
| | | ATGTGTGTCACCATCAGGCCAACAGGCCAGCCCTTGAATGGG |
| | | GATTTATTACTGTTGTATCTATGTTGCATGATAAACATTCATCA |
| | | CCTTCCTCCTGTAGTCCTGCCTCGTACTCCCCTTCCCCTATGAT |
| | | TGAAAAGTAAACAAAACCCACATTTCCTATCCTGGTTAGAAGA |
| | | AAATTAATGTTCTGACAGTTGTGATCGCCTGGAGTACTTTTAG |
| | | ACTTTTAGCATTCGTTTTTTACCTGTTTGTGGATGTGTGTTTGT |
| | | ATGTGCATACGTATGAGATAGGCACATGCATCTTCTGTATGGA |
| | | CAAAGGTGGGGTACCTACAGGAGAGCAAAGGTTAATTTTGTG |
| | | CTTTTAGTAAAAACATTTAAATACAAAGTTCTTTATTGGGTGG |
| | | AATTATATTTGATGCAAATATTTGATCACTTAAAACTTTTAAA |
| | | ACTTCTAGGTAATTTGCCACGCTTTTTGACTGCTCACCAATACC |
| | | CTGTAAAAATACGTAATTCTTCCTGTTTGTGTAATAAGATATTC |
| | | ATATTTGTAGTTGCATTAATAATAGTTATTTCTTAGTCCATCAG |
| | | ATGTTCCCGTGTGCCTCTTTTATGCCAAATTGATTGTCATATTT |
| | | CATGTTGGGACCAAGTAGTTTGCCCATGGCAAACCTAAATTTA |
| | | TGACCTGCTGAGGCCTCTCAGAAAACTGAGCATACTAGCAAG |
| | | ACAGCTCTTCTTGAAAAAAAAAATATGTATACACAAATATATA |
| | | CGTATATCTATATATACGTATGTATATACACACATGTATATTCT |
| | | TCCTTGATTGTGTAGCTGTCCAAAATAATAACATATATAGAGG |
| | | GAGCTGTATTCCTTTATACAAATCTGATGGCTCCTGCAGCACT |
| | | TTTTCCTTCTGAAAATATTTACATTTTGCTAACCTAGTTTGTTA |
| | | CTTTAAAAATCAGTTTTGATGAAAGGAGGGAAAAGCAGATGG |
| | | ACTTGAAAAAGATCCAAGCTCCTATTAGAAAAGGTATGAAAA |
| | | TCTTTATAGTAAAATTTTTTATAAACTAAAGTTGTACCTTTTAA |
| | | TATGTAGTAAACTCTCATTTATTTGGGGTTCGCTCTTGGATCTC |
| | | ATCCATCCATTGTGTTCTCTTTAATGCTGCCTGCCTTTTGAGGC |
| | | ATTCACTGCCCTAGACAATGCCACCAGAGATAGTGGGGGAAA |
| | | TGCCAGATGAAACCAACTCTTGCTCTCACTAGTTGTCAGCTTC |
| | | TCTGGATAAGTGACCACAGAAGCAGGAGTCCTCCTGCTTGGGC |
| | | ATCATTGGGCCAGTTCCTTCTCTTTAAATCAGATTTGTAATGGC |
| | | TCCCAAATTCCATCACATCACATTTAAATTGCAGACAGTGTTT |
| | | TGCACATCATGTATCTGTTTTGTCCCATAATATGCTTTTTACTC |
| | | CCTGATCCCAGTTTCTGCTGTTGACTCTTCCATTCAGTTTTATT |
| | | TATTGTGTGTTCTCACAGTGACACCATTTGTCCTTTTCTGCAAC |
| | | AACCTTTCCAGCTACTTTTGCCAAATTCTATTTGTCTTCTCCTT |
| | | CAAAACATTCTCCTTTGCAGTTCCTCTTCATCTGTGTAGCTGCT |
| | | CTTTTGTCTCTTAACTTACCATTCCTATAGTACTTTATGCATCT |
| | | CTGCTTAGTTCTATTAGTTTTTTGGCCTTGCTCTTCTCCTTGATT |
| | | TTAAAATTCCTTCTATAGCTAGAGCTTTTCTTTCTTTCATTCTCT |
| | | CTTCCTGCAGTGTTTTGCATACATCAGAAGCTAGGTACATAAG |
| | | TTAAATGATTGAGAGTTGGCTGTATTTAGATTTATCACTTTTTA |
| | | ATAGGGTGAGCTTGAGAGTTTTCTTTCTTTCTGTTTTTTTTTTTT |
| | | GTTTTTTTTTTTTTTTTTTTTTTTTTTTTTTTTGACTAATTTCACA |
| | | TGCTCTAAAAACCTTCAAAGGTGATTATTTTTCTCCTGGAAAC |
| | | TCCAGGTCCATTCTGTTTAAATCCCTAAGAATGTCAGAATTAA |
| | | AATAACAGGGCTATCCCGTAATTGGAAATATTTCTTTTTTCAG |
| | | GATGCTATAGTCAATTTAGTAAGTGACCACCAAATTGTTATTT |
| | | GCACTAACAAAGCTCAAAACACGATAAGTTTACTCCTCCATCT |
| | | CAGTAATAAAAATTAAGCTGTAATCAACCTTCTAGGTTTCTCT |
| | | TGTCTTAAAATGGGTATTCAAAAATGGGGATCTGTGGTGTATG |
| | | TATGGAAACACATACTCCTTAATTTACCTGTTGTTGGAAACTG |
| | | GAGAAATGATTGTCGGGCAACCGTTTATTTTTTATTGTATTTTA |
| | | TTTGGTTGAGGGATTTTTTTATAAACAGTTTTACTTGTGTCATA |
| | | TTTTAAAATTACTAACTGCCATCACCTGCTGGGGTCCTTTGTTA |
| | | GGTCATTTTCAGTGACTAATAGGGATAATCCAGGTAACTTTGA |
| | | AGAGATGAGCAGTGAGTGACCAGGCAGTTTTTCTGCCTTTAGC |
| | | TTTGACAGTTCTTAATTAAGATCATTGAAGACCAGCTTTCTCAT |
| | | AAATTTCTCTTTTTGAAAAAAAGAAAGCATTTGTACTAAGCTC |
| | | CTCTGTAAGACAACATCTTAAATCTTAAAAGTGTTGTTATCAT |
| | | GACTGGTGAGAGAAGAAAACATTTTGTTTTTATTAAATGGAGC |
| | | ATTATTTACAAAAAGCCATTGTTGAGAATTAGATCCCACATCG |
| | | TATAAATATCTATTAACCATTCTAAATAAAGAGAACTCCAGTG |
| | | TTGCTATGTGCAAGATCCTCTCTTGGAGCTTTTTTGCATAGCAA |
| | | TTAAAGGTGTGCTATTTGTCAGTAGCCATTTTTTTGCAGTGATT |
| | | TGAAGACCAAAGTTGTTTTACAGCTGTGTTACCGTTAAAGGTT |
| | | TTTTTTTTTATATGTATTAAATCAATTTATCACTGTTTAAAGCTT |
| | | TGAATATCTGCAATCTTTGCCAAGGTACTTTTTTATTTAAAAAA |
| | | AAACATAACTTTGTAAATATTACCCTGTAATATTATATATACTT |
| | | AATAAAACATTTTAAGCTATTTTGTTGGGCTATTTCTATTGCTG |
| | | CTACAGCAGACCACAAGCACATTTCTGAAAAATTTAATTTATT |
| | | AATGTATTTTTAAGTTGCTTATATTCTAGGTAACAATGTAAAG |
| | | AATGATTTAAAATATTAATTATGAATTTTTTGAGTATAATACCC |
| | | AATAAGCTTTTAATTAGAGCAGAGTTTTAATTAAAAGTTTTAA |
| | | ATCAGTC |
| leukocyte surface | 5 | MWPLAAALLLGSCCCGSAQLLFSNVNSIEFTSCNETVVIPCIVRN |
| antigen CD47 | | VEAQSTEEMFVKWKLNKSYIFIYDGNKNSTTTDQNFTSAKISVSD |
| isoform 4 precursor | | LINGIASLKMDKRDAMVGNYTCEVTELSREGKTVIELKNRTAFN |
| [Mus musculus] | | TDQGSACSYEEEKGGCKLVSWFSPNEKILIVIFPILAILLFWGKFGI |
| NCBI Reference | | LTLKYKSSHTNKRIILLLVAGLVLTVIVVVGAILLIPGEKPVKNAS |
| Sequence: | | GLGLIVISTGILILLQYNVFMTAFGMTSFTIAILITQVLGYVLALVG |
| NP_034711.1 | | LCLCIMACEPVHGPLLISGLGIIALAELLGLVYMKFVASNQRTIQP |
| | | PRNR |
| leukocyte surface | 6 | MWPLVAALLLGSACCGSAQLLFNKTKSVEFTFCNDTVVIPCFVT |
| antigen CD47 | | NMEAQNTTEVYVKWKFKGRDIYTFDGALNKSTVPTDFSSAKIEV |
| isoform 3 precursor | | SQLLKGDASLKMDKSDAVSHTGNYTCEVTELTREGETIIELKYRV |
| [Homo sapiens] | | VSWFSPNENILIVIFPIFAILLFWGQFGIKTLKYRSGGMDEKTIALL |
| NCBI Reference | | VAGLVITVIVIVGAILFVPGEYSLKNATGLGLIVTSTGILILLHYYV |
| NP_001369235.1 | | FSTAIGLTSFVIAILVIQVIAYILAVVGLSLCIAACIPMHGPLLISGLS |
| | | ILALAQLLGLVYMKFVASNQKTIQPPRKAVEEPLNE |
| leukocyte surface | 7 | MWPLVAALLLGSACCGSAQLLFNKTKSVEFTFCNDTVVIPCFVT |
| antigen CD47 | | NMEAQNTTEVYVKWKFKGRDIYTFDGALNKSTVPTDFSSAKIEV |
| isoform X2 [Homo | | SQLLKGDASLKMDKSDAVSHTGNYTCEVTELTREGETIIELKYRV |
| sapiens] | | VSWFSPNENILIVIFPIFAILLFWGQFGIKTLKYRSGGMDEKTIALL |
| XP_005247966.1 | | VAGLVITVIVIVGAILFVPGEYSLKNATGLGLIVTSTGILILLHYYV |
| | | FSTAIGLTSFVIAILVIQVIAYILAVVGLSLCIAACIPMHGPLLISGLS |
| | | ILALAQLLGLVYMKFVE |
| PREDICTED: Homo | 8 | GTGCGCGCGGCCGTGCAGCCTGGGCAGTGGGTCCTGCCTGTGA |
| sapiens CD47 | | CGCGCGGCGGCGGTCGGTCCTGCCTGTAACGGCGGCGGCGGC |
| molecule (CD47), | | TGCTGCTCCGGACACCTGCGGCGGCGGCGGCGACCCCGCGGC |
| transcript variant | | GGGCGCGGAGATGTGGCCCCTGGTAGCGGCGCTGTTGCTGGG |
| X11, mRNA | | CTCGGCGTGCTGCGGATCAGCTCAGCTACTATTTAATAAAACA |
| NCBI Reference | | AAATCTGTAGAATTCACGTTTTGTAATGACACTGTCGTCATTC |
| Sequence: | | CATGCTTTGTTACTAATATGGAGGCACAAAACACTACTGAAGT |
| XM_005247909.2 | | ATACGTAAAGTGGAAATTTAAAGGAAGAGATATTTACACCTTT |
| | | GATGGAGCTCTAAACAAGTCCACTGTCCCCACTGACTTTAGTA |
| | | GTGCAAAAATTGAAGTCTCACAATTACTAAAAGGAGATGCCT |
| | | CTTTGAAGATGGATAAGAGTGATGCTGTCTCACACACAGGAA |
| | | ACTACACTTGTGAAGTAACAGAATTAACCAGAGAAGGTGAAA |
| | | CGATCATCGAGCTAAAATATCGTGTTGTTTCATGGTTTTCTCCA |
| | | AATGAAAATATTCTTATTGTTATTTTCCCAATTTTTGCTATACT |
| | | CCTGTTCTGGGGACAGTTTGGTATTAAAACACTTAAATATAGA |
| | | TCCGGTGGTATGGATGAGAAAACAATTGCTTTACTTGTTGCTG |
| | | GACTAGTGATCACTGTCATTGTCATTGTTGGAGCCATTCTTTTC |
| | | GTCCCAGGTGAATATTCATTAAAGAATGCTACTGGCCTTGGTT |
| | | TAATTGTGACTTCTACAGGGATATTAATATTACTTCACTACTAT |
| | | GTGTTTAGTACAGCGATTGGATTAACCTCCTTCGTCATTGCCAT |
| | | ATTGGTTATTCAGGTGATAGCCTATATCCTCGCTGTGGTTGGA |
| | | CTGAGTCTCTGTATTGCGGCGTGTATACCAATGCATGGCCCTC |
| | | TTCTGATTTCAGGTTTGAGTATCTTAGCTCTAGCACAATTACTT |
| | | GGACTAGTTTATATGAAATTTGTGGAATAACTGAAGTGAAGTG |
| | | ATGGACTCCGATTTGGAGAGTAGTAAGACGTGAAAGGAATAC |
| | | ACTTGTGTTTAAGCACCATGGCCTTGATGATTCACTGTTGGGG |
| | | AGAAGAAACAAGAAAAGTAACTGGTTGTCACCTATGAGACCC |
| | | TTACGTGATTGTTAGTTAAGTTTTTATTCAAAGCAGCTGTAATT |
| | | TAGTTAATAAAATAATTATGATCTATGTTGTTTGCCCAATTGA |
| | | GATCCAGTTTTTTGTTGTTATTTTTAATCAATTAGGGGCAATAG |
| | | TAGAATGGACAATTTCCAAGAATGATGCCTTTCAGGTCCTAGG |
| | | GCCTCTGGCCTCTAGGTAACCAGTTTAAATTGGTTCAGGGTGA |
| | | TAACTACTTAGCACTGCCCTGGTGATTACCCAGAGATATCTAT |
| | | GAAAACCAGTGGCTTCCATCAAACCTTTGCCAACTCAGGTTCA |
| | | CAGCAGCTTTGGGCAGTTATGGCAGTATGGCATTAGCTGAGAG |
| | | GTGTCTGCCACTTCTGGGTCAATGGAATAATAAATTAAGTACA |
| | | GGCAGGAATTTGGTTGGGAGCATCTTGTATGATCTCCGTATGA |
| | | TGTGATATTGATGGAGATAGTGGTCCTCATTCTTGGGGGTTGC |
| | | CATTCCCACATTCCCCCTTCAACAAACAGTGTAACAGGTCCTT |
| | | CCCAGATTTAGGGTACTTTTATTGATGGATATGTTTTCCTTTTA |
| | | TTCACATAACCCCTTGAAACCCTGTCTTGTCCTCCTGTTACTTG |
| | | CTTCTGCTGTACAAGATGTAGCACCTTTTCTCCTCTTTGAACAT |
| | | GGTCTAGTGACACGGTAGCACCAGTTGCAGGAAGGAGCCAGA |
| | | CTTGTTCTCAGAGCACTGTGTTCACACTTTTCAGCAAAAATAG |
| | | CTATGGTTGTAACATATGTATTCCCTTCCTCTGATTTGAAGGCA |
| | | AAAATCTACAGTGTTTCTTCACTTCTTTTCTGATCTGGGGCATG |
| | | AAAAAAGCAAGATTGAAATTTGAACTATGAGTCTCCTGCATG |
| | | GCAACAAAATGTGTGTCACCATCAGGCCAACAGGCCAGCCCT |
| | | TGAATGGGGATTTATTACTGTTGTATCTATGTTGCATGATAAA |
| | | CATTCATCACCTTCCTCCTGTAGTCCTGCCTCGTACTCCCCTTC |
| | | CCCTATGATTGAAAAGTAAACAAAACCCACATTTCCTATCCTG |
| | | GTTAGAAGAAAATTAATGTTCTGACAGTTGTGATCGCCTGGAG |
| | | TACTTTTAGACTTTTAGCATTCGTTTTTTACCTGTTTGTGGATGT |
| | | GTGTTTGTATGTGCATACGTATGAGATAGGCACATGCATCTTC |
| | | TGTATGGACAAAGGTGGGGTACCTACAGGAGAGCAAAGGTTA |
| | | ATTTTGTGCTTTTAGTAAAAACATTTAAATACAAAGTTCTTTAT |
| | | TGGGTGGAATTATATTTGATGCAAATATTTGATCACTTAAAAC |
| | | TTTTAAAACTTCTAGGTAATTTGCCACGCTTTTTGACTGCTCAC |
| | | CAATACCCTGTAAAAATACGTAATTCTTCCTGTTTGTGTAATA |
| | | AGATATTCATATTTGTAGTTGCATTAATAATAGTTATTTCTTAG |
| | | TCCATCAGATGTTCCCGTGTGCCTCTTTTATGCCAAATTGATTG |
| | | TCATATTTCATGTTGGGACCAAGTAGTTTGCCCATGGCAAACC |
| | | TAAATTTATGACCTGCTGAGGCCTCTCAGAAAACTGAGCATAC |
| | | TAGCAAGACAGCTCTTCTTGAAAAAAAAAATATGTATACACA |
| | | AATATATACGTATATCTATATATACGTATGTATATACACACAT |
| | | GTATATTCTTCCTTGATTGTGTAGCTGTCCAAAATAATAACAT |
| | | ATATAGAGGGAGCTGTATTCCTTTATACAAATCTGATGGCTCC |
| | | TGCAGCACTTTTTCCTTCTGAAAATATTTACATTTTGCTAACCT |
| | | AGTTTGTTACTTTAAAAATCAGTTTTGATGAAAGGAGGGAAAA |
| | | GCAGATGGACTTGAAAAAGATCCAAGCTCCTATTAGAAAAGG |
| | | TATGAAAATCTTTATAGTAAAATTTTTTATAAACTAAAGTTGT |
| | | ACCTTTTAATATGTAGTAAACTCTCATTTATTTGGGGTTCGCTC |
| | | TTGGATCTCATCCATCCATTGTGTTCTCTTTAATGCTGCCTGCC |
| | | TTTTGAGGCATTCACTGCCCTAGACAATGCCACCAGAGATAGT |
| | | GGGGGAAATGCCAGATGAAACCAACTCTTGCTCTCACTAGTTG |
| | | TCAGCTTCTCTGGATAAGTGACCACAGAAGCAGGAGTCCTCCT |
| | | GCTTGGGCATCATTGGGCCAGTTCCTTCTCTTTAAATCAGATTT |
| | | GTAATGGCTCCCAAATTCCATCACATCACATTTAAATTGCAGA |
| | | CAGTGTTTTGCACATCATGTATCTGTTTTGTCCCATAATATGCT |
| | | TTTTACTCCCTGATCCCAGTTTCTGCTGTTGACTCTTCCATTCA |
| | | GTTTTATTTATTGTGTGTTCTCACAGTGACACCATTTGTCCTTT |
| | | TCTGCAACAACCTTTCCAGCTACTTTTGCCAAATTCTATTTGTC |
| | | TTCTCCTTCAAAACATTCTCCTTTGCAGTTCCTCTTCATCTGTG |
| | | TAGCTGCTCTTTTGTCTCTTAACTTACCATTCCTATAGTACTTT |
| | | ATGCATCTCTGCTTAGTTCTATTAGTTTTTTGGCCTTGCTCTTCT |
| | | CCTTGATTTTAAAATTCCTTCTATAGCTAGAGCTTTTCTTTCTTT |
| | | CATTCTCTCTTCCTGCAGTGTTTTGCATACATCAGAAGCTAGGT |
| | | ACATAAGTTAAATGATTGAGAGTTGGCTGTATTTAGATTTATC |
| | | ACTTTTTAATAGGGTGAGCTTGAGAGTTTTCTTTCTTTCTGTTT |
| | | TTTTTTTTTGTTTTTTTTTTTTTTTTTTTTTTTTTTTTTTTTGACTA |
| | | ATTTCACATGCTCTAAAAACCTTCAAAGGTGATTATTTTTCTCC |
| | | TGGAAACTCCAGGTCCATTCTGTTTAAATCCCTAAGAATGTCA |
| | | GAATTAAAATAACAGGGCTATCCCGTAATTGGAAATATTTCTT |
| | | TTTTCAGGATGCTATAGTCAATTTAGTAAGTGACCACCAAATT |
| | | GTTATTTGCACTAACAAAGCTCAAAACACGATAAGTTTACTCC |
| | | TCCATCTCAGTAATAAAAATTAAGCTGTAATCAACCTTCTAGG |
| | | TTTCTCTTGTCTTAAAATGGGTATTCAAAAATGGGGATCTGTG |
| | | GTGTATGTATGGAAACACATACTCCTTAATTTACCTGTTGTTG |
| | | GAAACTGGAGAAATGATTGTCGGGCAACCGTTTATTTTTTATT |
| | | GTATTTTATTTGGTTGAGGGATTTTTTTATAAACAGTTTTACTT |
| | | GTGTCATATTTTAAAATTACTAACTGCCATCACCTGCTGGGGT |
| | | CCTTTGTTAGGTCATTTTCAGTGACTAATAGGGATAATCCAGG |
| | | TAACTTTGAAGAGATGAGCAGTGAGTGACCAGGCAGTTTTTCT |
| | | GCCTTTAGCTTTGACAGTTCTTAATTAAGATCATTGAAGACCA |
| | | GCTTTCTCATAAATTTCTCTTTTTGAAAAAAAGAAAGCATTTGT |
| | | ACTAAGCTCCTCTGTAAGACAACATCTTAAATCTTAAAAGTGT |
| | | TGTTATCATGACTGGTGAGAGAAGAAAACATTTTGTTTTTATT |
| | | AAATGGAGCATTATTTACAAAAAGCCATTGTTGAGAATTAGAT |
| | | CCCACATCGTATAAATATCTATTAACCATTCTAAATAAAGAGA |
| | | ACTCCAGTGTTGCTATGTGCAAGATCCTCTCTTGGAGCTTTTTT |
| | | GCATAGCAATTAAAGGTGTGCTATTTGTCAGTAGCCATTTTTTT |
| | | GCAGTGATTTGAAGACCAAAGTTGTTTTACAGCTGTGTTACCG |
| | | TTAAAGGTTTTTTTTTTTATATGTATTAAATCAATTTATCACTG |
| | | TTTAAAGCTTTGAATATCTGCAATCTTTGCCAAGGTACTTTTTT |
| | | ATTTAAAAAAAAACATAACTTTGTAAATATTACCCTGTAATAT |
| | | TATATATACTTAATAAAACATTTTAAGCTA |
| Homo sapiens CD47 | 9 | GCAGCCTGGGCAGTGGGTCCTGCCTGTGACGCGCGGCGGCGG |
| molecule (CD47), | | TCGGTCCTGCCTGTAACGGCGGCGGCGGCTGCTGCTCCGGACA |
| transcript variant 3, | | CCTGCGGCGGCGGCGGCGACCCCGCGGCGGGCGCGGAGATGT |
| mRNA | | GGCCCCTGGTAGCGGCGCTGTTGCTGGGCTCGGCGTGCTGCGG |
| NCBI Reference | | ATCAGCTCAGCTACTATTTAATAAAACAAAATCTGTAGAATTC |
| Sequence: | | ACGTTTTGTAATGACACTGTCGTCATTCCATGCTTTGTTACTAA |
| NM_001382306.1 | | TATGGAGGCACAAAACACTACTGAAGTATACGTAAAGTGGAA |
| | | ATTTAAAGGAAGAGATATTTACACCTTTGATGGAGCTCTAAAC |
| | | AAGTCCACTGTCCCCACTGACTTTAGTAGTGCAAAAATTGAAG |
| | | TCTCACAATTACTAAAAGGAGATGCCTCTTTGAAGATGGATAA |
| | | GAGTGATGCTGTCTCACACACAGGAAACTACACTTGTGAAGTA |
| | | ACAGAATTAACCAGAGAAGGTGAAACGATCATCGAGCTAAAA |
| | | TATCGTGTTGTTTCATGGTTTTCTCCAAATGAAAATATTCTTAT |
| | | TGTTATTTTCCCAATTTTTGCTATACTCCTGTTCTGGGGACAGT |
| | | TTGGTATTAAAACACTTAAATATAGATCCGGTGGTATGGATGA |
| | | GAAAACAATTGCTTTACTTGTTGCTGGACTAGTGATCACTGTC |
| | | ATTGTCATTGTTGGAGCCATTCTTTTCGTCCCAGGTGAATATTC |
| | | ATTAAAGAATGCTACTGGCCTTGGTTTAATTGTGACTTCTACA |
| | | GGGATATTAATATTACTTCACTACTATGTGTTTAGTACAGCGA |
| | | TTGGATTAACCTCCTTCGTCATTGCCATATTGGTTATTCAGGTG |
| | | ATAGCCTATATCCTCGCTGTGGTTGGACTGAGTCTCTGTATTGC |
| | | GGCGTGTATACCAATGCATGGCCCTCTTCTGATTTCAGGTTTG |
| | | AGTATCTTAGCTCTAGCACAATTACTTGGACTAGTTTATATGA |
| | | AATTTGTGGCTTCCAATCAGAAGACTATACAACCTCCTAGGAA |
| | | AGCTGTAGAGGAACCCCTTAATGAATAACTGAAGTGAAGTGA |
| | | TGGACTCCGATTTGGAGAGTAGTAAGACGTGAAAGGAATACA |
| | | CTTGTGTTTAAGCACCATGGCCTTGATGATTCACTGTTGGGGA |
| | | GAAGAAACAAGAAAAGTAACTGGTTGTCACCTATGAGACCCT |
| | | TACGTGATTGTTAGTTAAGTTTTTATTCAAAGCAGCTGTAATTT |
| | | AGTTAATAAAATAATTATGATCTATGTTGTTTGCCCAATTGAG |
| | | ATCCAGTTTTTTGTTGTTATTTTTAATCAATTAGGGGCAATAGT |
| | | AGAATGGACAATTTCCAAGAATGATGCCTTTCAGGTCCTAGGG |
| | | CCTCTGGCCTCTAGGTAACCAGTTTAAATTGGTTCAGGGTGAT |
| | | AACTACTTAGCACTGCCCTGGTGATTACCCAGAGATATCTATG |
| | | AAAACCAGTGGCTTCCATCAAACCTTTGCCAACTCAGGTTCAC |
| | | AGCAGCTTTGGGCAGTTATGGCAGTATGGCATTAGCTGAGAG |
| | | GTGTCTGCCACTTCTGGGTCAATGGAATAATAAATTAAGTACA |
| | | GGCAGGAATTTGGTTGGGAGCATCTTGTATGATCTCCGTATGA |
| | | TGTGATATTGATGGAGATAGTGGTCCTCATTCTTGGGGGTTGC |
| | | CATTCCCACATTCCCCCTTCAACAAACAGTGTAACAGGTCCTT |
| | | CCCAGATTTAGGGTACTTTTATTGATGGATATGTTTTCCTTTTA |
| | | TTCACATAACCCCTTGAAACCCTGTCTTGTCCTCCTGTTACTTG |
| | | CTTCTGCTGTACAAGATGTAGCACCTTTTCTCCTCTTTGAACAT |
| | | GGTCTAGTGACACGGTAGCACCAGTTGCAGGAAGGAGCCAGA |
| | | CTTGTTCTCAGAGCACTGTGTTCACACTTTTCAGCAAAAATAG |
| | | CTATGGTTGTAACATATGTATTCCCTTCCTCTGATTTGAAGGCA |
| | | AAAATCTACAGTGTTTCTTCACTTCTTTTCTGATCTGGGGCATG |
| | | AAAAAAGCAAGATTGAAATTTGAACTATGAGTCTCCTGCATG |
| | | GCAACAAAATGTGTGTCACCATCAGGCCAACAGGCCAGCCCT |
| | | TGAATGGGGATTTATTACTGTTGTATCTATGTTGCATGATAAA |
| | | CATTCATCACCTTCCTCCTGTAGTCCTGCCTCGTACTCCCCTTC |
| | | CCCTATGATTGAAAAGTAAACAAAACCCACATTTCCTATCCTG |
| | | GTTAGAAGAAAATTAATGTTCTGACAGTTGTGATCGCCTGGAG |
| | | TACTTTTAGACTTTTAGCATTCGTTTTTTACCTGTTTGTGGATGT |
| | | GTGTTTGTATGTGCATACGTATGAGATAGGCACATGCATCTTC |
| | | TGTATGGACAAAGGTGGGGTACCTACAGGAGAGCAAAGGTTA |
| | | ATTTTGTGCTTTTAGTAAAAACATTTAAATACAAAGTTCTTTAT |
| | | TGGGTGGAATTATATTTGATGCAAATATTTGATCACTTAAAAC |
| | | TTTTAAAACTTCTAGGTAATTTGCCACGCTTTTTGACTGCTCAC |
| | | CAATACCCTGTAAAAATACGTAATTCTTCCTGTTTGTGTAATA |
| | | AGATATTCATATTTGTAGTTGCATTAATAATAGTTATTTCTTAG |
| | | TCCATCAGATGTTCCCGTGTGCCTCTTTTATGCCAAATTGATTG |
| | | TCATATTTCATGTTGGGACCAAGTAGTTTGCCCATGGCAAACC |
| | | TAAATTTATGACCTGCTGAGGCCTCTCAGAAAACTGAGCATAC |
| | | TAGCAAGACAGCTCTTCTTGAAAAAAAAAATATGTATACACA |
| | | AATATATACGTATATCTATATATACGTATGTATATACACACAT |
| | | GTATATTCTTCCTTGATTGTGTAGCTGTCCAAAATAATAACAT |
| | | ATATAGAGGGAGCTGTATTCCTTTATACAAATCTGATGGCTCC |
| | | TGCAGCACTTTTTCCTTCTGAAAATATTTACATTTTGCTAACCT |
| | | AGTTTGTTACTTTAAAAATCAGTTTTGATGAAAGGAGGGAAAA |
| | | GCAGATGGACTTGAAAAAGATCCAAGCTCCTATTAGAAAAGG |
| | | TATGAAAATCTTTATAGTAAAATTTTTTATAAACTAAAGTTGT |
| | | ACCTTTTAATATGTAGTAAACTCTCATTTATTTGGGGTTCGCTC |
| | | TTGGATCTCATCCATCCATTGTGTTCTCTTTAATGCTGCCTGCC |
| | | TTTTGAGGCATTCACTGCCCTAGACAATGCCACCAGAGATAGT |
| | | GGGGGAAATGCCAGATGAAACCAACTCTTGCTCTCACTAGTTG |
| | | TCAGCTTCTCTGGATAAGTGACCACAGAAGCAGGAGTCCTCCT |
| | | GCTTGGGCATCATTGGGCCAGTTCCTTCTCTTTAAATCAGATTT |
| | | GTAATGGCTCCCAAATTCCATCACATCACATTTAAATTGCAGA |
| | | CAGTGTTTTGCACATCATGTATCTGTTTTGTCCCATAATATGCT |
| | | TTTTACTCCCTGATCCCAGTTTCTGCTGTTGACTCTTCCATTCA |
| | | GTTTTATTTATTGTGTGTTCTCACAGTGACACCATTTGTCCTTT |
| | | TCTGCAACAACCTTTCCAGCTACTTTTGCCAAATTCTATTTGTC |
| | | TTCTCCTTCAAAACATTCTCCTTTGCAGTTCCTCTTCATCTGTG |
| | | TAGCTGCTCTTTTGTCTCTTAACTTACCATTCCTATAGTACTTT |
| | | ATGCATCTCTGCTTAGTTCTATTAGTTTTTTGGCCTTGCTCTTCT |
| | | CCTTGATTTTAAAATTCCTTCTATAGCTAGAGCTTTTCTTTCTTT |
| | | CATTCTCTCTTCCTGCAGTGTTTTGCATACATCAGAAGCTAGGT |
| | | ACATAAGTTAAATGATTGAGAGTTGGCTGTATTTAGATTTATC |
| | | ACTTTTTAATAGGGTGAGCTTGAGAGTTTTCTTTCTTTCTGTTT |
| | | TTTTTTTTTGTTTTTTTTTTTTTTTTTTTTTTTTTTTTTTTTGACTA |
| | | ATTTCACATGCTCTAAAAACCTTCAAAGGTGATTATTTTTCTCC |
| | | TGGAAACTCCAGGTCCATTCTGTTTAAATCCCTAAGAATGTCA |
| | | GAATTAAAATAACAGGGCTATCCCGTAATTGGAAATATTTCTT |
| | | TTTTCAGGATGCTATAGTCAATTTAGTAAGTGACCACCAAATT |
| | | GTTATTTGCACTAACAAAGCTCAAAACACGATAAGTTTACTCC |
| | | TCCATCTCAGTAATAAAAATTAAGCTGTAATCAACCTTCTAGG |
| | | TTTCTCTTGTCTTAAAATGGGTATTCAAAAATGGGGATCTGTG |
| | | GTGTATGTATGGAAACACATACTCCTTAATTTACCTGTTGTTG |
| | | GAAACTGGAGAAATGATTGTCGGGCAACCGTTTATTTTTTATT |
| | | GTATTTTATTTGGTTGAGGGATTTTTTTATAAACAGTTTTACTT |
| | | GTGTCATATTTTAAAATTACTAACTGCCATCACCTGCTGGGGT |
| | | CCTTTGTTAGGTCATTTTCAGTGACTAATAGGGATAATCCAGG |
| | | TAACTTTGAAGAGATGAGCAGTGAGTGACCAGGCAGTTTTTCT |
| | | GCCTTTAGCTTTGACAGTTCTTAATTAAGATCATTGAAGACCA |
| | | GCTTTCTCATAAATTTCTCTTTTTGAAAAAAAGAAAGCATTTGT |
| | | ACTAAGCTCCTCTGTAAGACAACATCTTAAATCTTAAAAGTGT |
| | | TGTTATCATGACTGGTGAGAGAAGAAAACATTTTGTTTTTATT |
| | | AAATGGAGCATTATTTACAAAAAGCCATTGTTGAGAATTAGAT |
| | | CCCACATCGTATAAATATCTATTAACCATTCTAAATAAAGAGA |
| | | ACTCCAGTGTTGCTATGTGCAAGATCCTCTCTTGGAGCTTTTTT |
| | | GCATAGCAATTAAAGGTGTGCTATTTGTCAGTAGCCATTTTTTT |
| | | GCAGTGATTTGAAGACCAAAGTTGTTTTACAGCTGTGTTACCG |
| | | TTAAAGGTTTTTTTTTTTATATGTATTAAATCAATTTATCACTG |
| | | TTTAAAGCTTTGAATATCTGCAATCTTTGCCAAGGTACTTTTTT |
| | | ATTTAAAAAAAAACATAACTTTGTAAATATTACCCTGTAATAT |
| | | TATATATACTTAATAAAACATTTTAAGCTATTTTGTTGGGCTAT |
| | | TTCTATTGCTGCTACAGCAGACCACAAGCACATTTCTGAAAAA |
| | | TTTAATTTATTAATGTATTTTTAAGTTGCTTATATTCTAGGTAA |
| | | CAATGTAAAGAATGATTTAAAATATTAATTATGAATTTTTTGA |
| | | GTATAATACCCAATAAGCTTTTAATTAGAGCAGAGTTTTAATT |
| | | AAAAGTTTTAAATCAGTCCAA |

### 9. EXAMPLES

The examples in this Section (i.e., Section 9) are offered by way of illustration, and not by way of limitation.

### 9.1 Example 1: Human CD47 Expression on Glomerular Cells Correlates with Avoidance of Proteinuria via the Human CD47-SIRPα Pathway

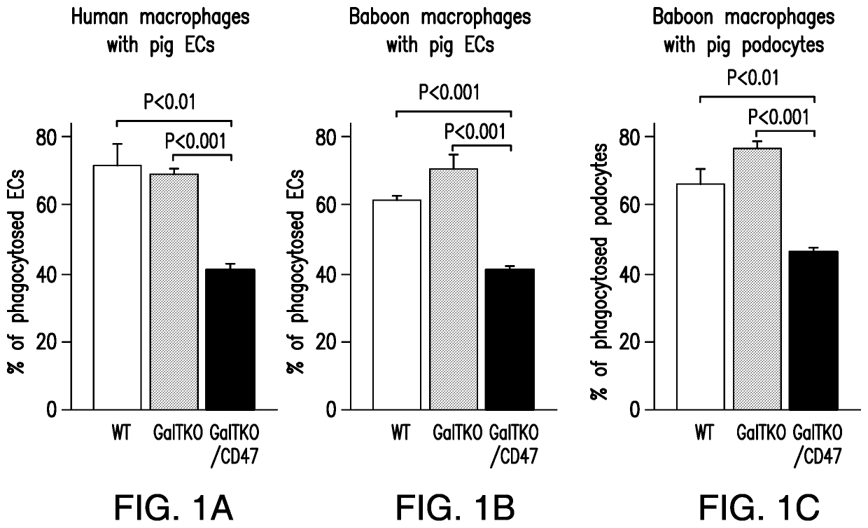

It was examined whether baboon macrophages phagocytosed porcine endothelial cells (ECs) similarly to human macrophages. We found that both human and baboon macrophages phagocytosed porcine ECs similarly. Strikingly, this response was significantly reduced when porcine ECs and podocytes expressed human CD47/human CD55 but not human CD46/human CD55 without human CD47 (FIG. 1A-FIG. 1C). Using grafts from human CD47/human CD55 Tg GalT-KO donors, we found that GalT-KO porcine kidneys with high expression of human-CD47 on the glomerular cells minimized the development of proteinuria even without CTLA4-Ig treatment.

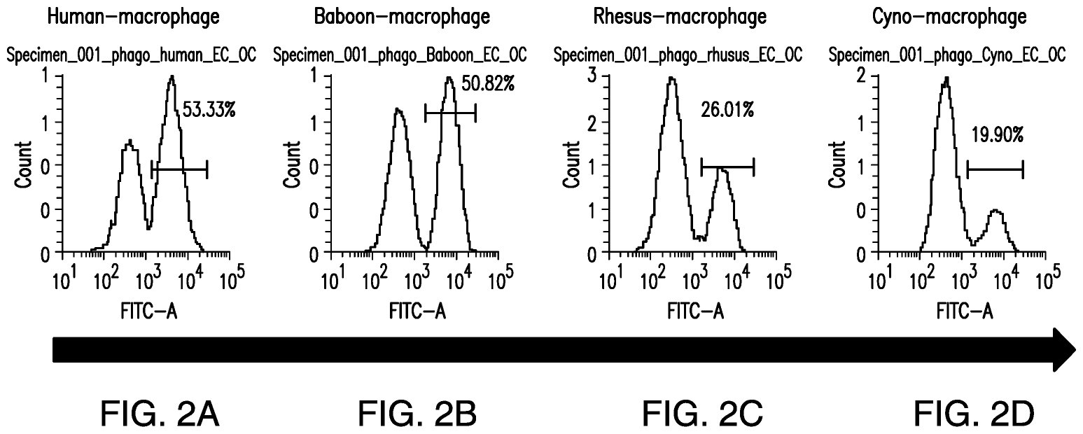

We further examined phagocytosis of GalT-KO ECs using human, baboon, rhesus, and cynomolgus macrophages. While human and baboon macrophages phagocytosed pig ECs and podocytes similarly and aggressively, rhesus and cynomolgus macaque macrophages phagocytosed GalTKO EC markedly less than observed for baboon or human macrophages (FIG. 2A-FIG. 2D).

The above discussed findings indicate that species incompatibility between pig and baboon plays an essential role in the development of post-xeno KTx proteinuria and will be relevant in humans and that a strategy to prevent the development of proteinuria is essential for the success of pig to human xenotransplantation.

### 9.2 Example 2: High Human CD47 Expression on Tubular Cells Results in Edema Associated with TSP-1 Upregulation

While CD47 is known to bind SIRPα and block its activation, CD47 also binds to TSP1 (CD47-TSP-1 pathway) which inhibits nitric oxide signaling in vascular cells and induces activation of the innate immune response and cell proliferation or apoptosis. In our vascularized thymic lobe plus kidney xenotransplantation (“VT+K XTx”) model, a baboon that received a GalT-KO kidney with a glomerular cell-specific expression of human CD47 maintained the renal xenograft without CTLA4-Ig for 128 days (until the graft outgrew the available space) without evidence of rejection or proteinuria. In contrast, baboons that received VT+K grafts with high expression of human CD47 on all cells, including renal tubular cells, were euthanized due to systemic subcutaneous and tracheal edema without an increase in serum Cre or proteinuria. These baboons further also demonstrated high levels of chimerism (15-30% T cell chimerism) in the first post-op week. Subsequently, systemic edema developed, and IL-6 levels increased in serum. Excised kidney grafts at POD 50 and 53 showed tubular atrophy, and interstitial cell infiltrates, suggesting that TSP-1 mediated inflammatory responses in the kidney grafts. Notably, the media layers of blood vessels of kidney grafts showed upregulation of TSP-1. Systemic edema was also found to be accompanied by elevated IL-6 levels in serum.

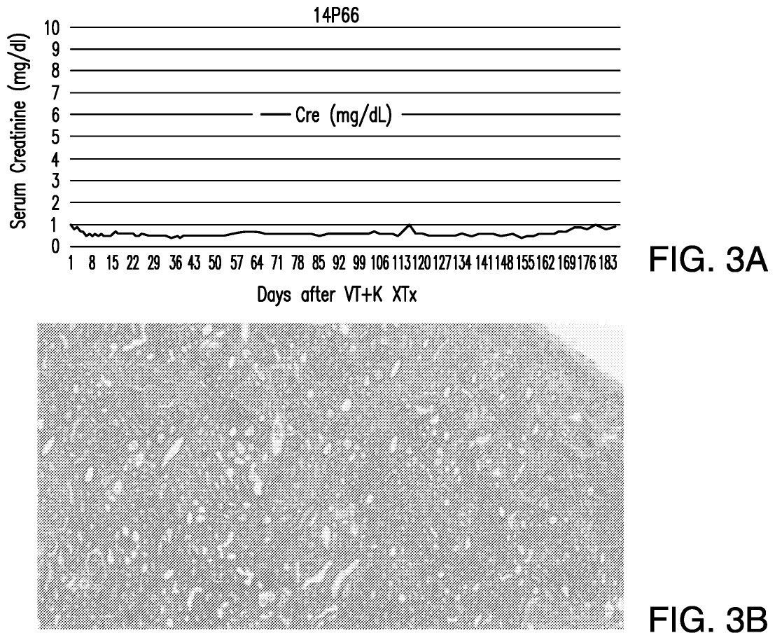

Based upon these findings, we added anti-IL6r antibody once a week until POD 42. Anti-IL-6R ab seems to have inhibited inflammatory changes and extended the survival of baboons without the early inflammatory events or proteinuria. While the exponential growth of pig grafts or drug-related side effects triggered the euthanasia of recipient baboons, we confirmed no apparent signs of graft rejection, pig-specific unresponsiveness in vitro, and the development of new baboon T cells at the above time points. FIG. 3 shows sCre levels (FIG. 3A) and histologic findings (FIG. 3B) of an excised kidney graft at POD 187.

### 9.3 Example 3—Podocyte-Specific Expression of Human CD47 Gene in Miniature Swine

This example provides a method of construction of a miniature swine expressing human CD47 under control of a podocyte-specific promoter, namely, the nephrin promoter.

Fibroblasts containing a random integration of a vector consisting of the human CD47 expressed from the pig nephrin promoter (FIG. 4) will be selected. The promoter region of this vector will include the upstream portions of the adjacent Kirrel2 promoter and therefore will contain all elements required from tissue-specific expression. To increase the likelihood for specific expression, intron 1 and a short segment of exon 2 of the nephrin gene will also be included, with sequences coding for the mature form of human CD47 joined to the resulting nephrin leader peptide. Selection for cells which have integrated the vector into transcriptionally permissive genomic locations will be on the basis of GFP expression from the ubiquitous PGK promoter.

Screening for appropriate expression of the human CD47 gene will be performed in 2nd trimester cloned fetuses. Widespread expression of GFP is expected. However, human CD47 expression (as measured on cell surface, and/or by RNA analysis) will be limited to the kidney in desired clones. Fibroblasts isolated from fetuses with the desired expression profile will be used to generate pigs in a second round of nuclear transfer.

Kidneys from these pigs will be evaluated in baboon transplants. These animals are tested for TSP1 activation (as measured by RT-PCR). These animals are also tested for proteinuria.

### 9.4 Example 4—Effect of Glomeruli-Specific Expression on Proteinuria

To show the effect of glomeruli-specific expression of human CD47 on xenograft tolerance, miniature swine expressing human CD47 specifically in the glomeruli of the kidney are generated. Kidney from these swine are transplanted into baboons, along with bone marrow from a different miniature swine which also expresses human CD47. As a comparison, kidneys and bone marrow from swine which ubiquitously express human CD47, or kidneys and bone marrow from swine which express human CD47 in the bone marrow but not the kidney, are transplanted into baboons. Proteinuria is assessed by measuring urinary protein concentration after transplanting.

### 10. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

1. A method for preventing or reducing the severity of proteinuria in a kidney transplant recipient, wherein the method comprises:
(a) transplanting into the recipient a kidney, wherein the kidney is obtained from an alpha-1,3 galactosyltransferase-deficient miniature swine and glomeruli of the kidney express human CD47 at levels sufficient to prevent or reduce the severity of proteinuria in the recipient; and
(b) transplanting into the recipient porcine hematopoietic stem cells, wherein the porcine hematopoietic stem cells express human CD47 and are obtained from an alpha-1,3 galactosyltransferase-deficient miniature swine.

2. The method of claim 1, wherein the glomeruli of the kidney express human CD47 at a level higher than the level of human CD47 expression in the tubules of the kidney.

3. The method of claim 2, wherein the glomeruli of the kidney express human CD47 at a level 2 times to 10 times higher than the level of human CD47 expression in the tubules of the kidney.

4. The method of claim 1, wherein the alpha-1,3 galactosyltransferase-deficient miniature swine is a MHC-inbred Columbia/Sachs miniature swine.

5. The method of claim 1, wherein the level of human CD47 expression is measured by real-time polymerase chain reaction.

6. The method of claim 1, wherein the recipient is a mammal.

7. The method of claim 6, wherein the recipient is a human,

8. The method of claim 1, wherein the porcine hematopoietic stem cells are obtained from bone marrow, peripheral blood, umbilical cord blood, or fetal liver cells.

9. The method of claim 1, wherein the human CD47 is expressed under the same regulatory elements as the endogenous porcine CD47.

10. The method of claim 1, wherein the human CD47 replaces an endogenous porcine CD47 in the alpha-1,3 galactosyltransferase-deficient miniature swine.

11. The method of claim 1, wherein the human CD47 is expressed under a glomerulus-specific promoter.

12. The method of claim 11, wherein the glomerulus-specific promoter is nephrin.

13. The method of claim 1, wherein the proteinuria:
(a) is renal proteinuria;
(b) is reduced to:
(i) less than 3 g per 24 hours
(ii) 500 mg per 24 hours;
(iii) 300 mg per 24 hours; or
(iv) 150 mg per 24 hours; and/or
(c) resolves within:
(i) two weeks of the transplant.
(ii) one month of the transplant.
(iii) two months of the transplant or
(iv) four months of the transplant.

14.-21. (canceled)

22. The method of claim 1, wherein the kidney is a thymokidney.

23. A kidney isolated from a miniature swine, wherein the glomeruli of the kidney express human CD47 at a level higher than the level of human CD47 expression in the tubules of the kidney, optionally wherein the glomeruli of the kidney express human CD47 at a level 2 times to 10 times higher than the level of human CD47 expression in the tubules of the kidney.

24. (canceled)

25. The kidney of claim 23, wherein the level of human CD47 expression is measured by real-time polymerase chain reaction.

26. The kidney of claim 23, wherein the human CD47 is expressed under:
(a) the same regulatory elements as the endogenous porcine CD47: and/or
(b) under a glomerulus-specific promoter, optionally wherein the glomerulus-specific promoter is nephrin.

27.-28. (canceled)

29. The kidney of claim 23, wherein the kidney is a thymokidney.

30. The kidney of claim 23, wherein the miniature swine is an alpha-1,3 galactosyltransferase-deficient miniature swine, optionally wherein the alpha-1,3 galactosyltransferase-deficient miniature swine is a MHC-inbred Columbia/Sachs miniature swine.

31. (canceled)

32. A method of transplanting a kidney from a miniature swine into a human recipient, wherein the method comprises:
(a) transplanting bone marrow from a first miniature swine to the recipient via intrabone transplantation; and
(b) transplanting a kidney from a second miniature swine to the recipient.

33. The method of claim 32, wherein said second step of transplanting a kidney from a second miniature swine is carried out at least 28 days after first step of transplanting bone marrow from a first miniature swine.

34. The method of claim 32, wherein:
(a) the bone marrow from the first miniature swine expresses human CD47; and/or
(b) the kidney from the second miniature swine expresses human CD47.

35-36. (canceled)

37. The method of claim 34, wherein the human CD47 is expressed under:
(a) the same regulatory elements as the endogenous porcine CD47; and/or
(b) a glomerulus-specific promoter, optionally wherein the human CD47 is expressed under a glomerulus-specific promoter.

38.-39. (canceled)

40. The method of claim 32, wherein the bone marrow and the kidney are from the same miniature swine.

41. The method of claim 32, wherein the first miniature swine and the second miniature swine are:
(a) from the same, highly inbred herd of miniature swine;
(b) alpha-1,3 galactosyltransferase-deficient miniature swine, optionally wherein the alpha-1,3 galactosyltransferase-deficient miniature swine are MHC-inbred Columbia/Sachs miniature swine;
(c) genetically matched miniature swine; and/or
(d) MHC matched.

42.-45. (canceled)

46. The method of claim 32, further comprising:
(a) administration of one or more additional treatments to the recipient, optionally wherein the one or more additional treatment is selected from the group comprising total body irradiation, thymic irradiation, rituximab, anti-thymocyte globulin (ATG), tacrolimus, mycophenolate mofetil (MMF), anti-CD154 antibodies, cobra venom factor (CVF), heparin, prostacyclin, recombinant porcine cytokines, porcine stem cell factor (pCSF), porcine interleukin-3 (pIL-3), ganciclovir, methylprednisolone, anti-IL6 receptor antibodies and anti-CD40 antibodies;
(b) transplanting islet of Langerhans cells from a miniature swine to the recipient.

47.-48. (canceled)

49. A xenograft from a non-human species, wherein the xenograft comprises:
(a) a kidney; and
(b) islet of Langerhans cells,
wherein the kidney comprises glomeruli that express human CD47 at a level higher than the level of human CD47 expression in the tubules of the kidney, optionally wherein the glomeruli of the kidney express human CD47 at a level 2 times to 10 times higher than the level of human CD47 expression in the tubules of the kidney.

50. (canceled)

51. The xenograft of claim 49, wherein the level of human CD47 expression is measured by real-time polymerase chain reaction.

52. The xenograft of claim 49, wherein the human CD47 is expressed under:
(a) the same regulatory elements as the endogenous porcine CD47; and/or
(b) a glomerulus-specific promoter, optionally wherein the glomerulus-specific promoter is nephrin.

53.-54. (canceled)

55. The xenograft of claim 49, wherein the kidney is a thymokidney.
